SEQUENCE LISTING (43 sequences)

SEQ ID NO: 1 - DNA, 32 bp, Artificial Sequence
  Fab 1 ss oligo
ttttttttttttctttgttcttattctctgct

SEQ ID NO: 2 - DNA, 32 bp, artificial sequence
  Fab 2 ssoligo
aagagagagaaaaggaagaagggaagaagagg

SEQ ID NO: 3 - DNA, 22 bp, Artificial Sequence
  10 bp 1 linker
ttttttttttttggacgaagtc

SEQ ID NO: 4 - DNA, 22 bp, artificial sequence
  10bp 2 linker
aagagagagaaagacttcgtcc

SEQ ID NO: 5 - DNA, 27 bp, artificial sequence
  15bp 1 linker
ttttttttttttggacgaagtccaacc

SEQ ID NO: 6 - DNA, 27 bp, artificial sequence
  15bp 2 linker
aagagagagaaaggttggacttcgtcc

SEQ ID NO: 7 - DNA, 32 bp, artificial sequence
  20bp 1 linker
ttttttttttttcgtggtcatgagccgggacg

SEQ ID NO: 8 - DNA, 32 bp, artificial sequence
  20bp 2 linker
aagagagagaaacgtcccggctcatgaccacg

SEQ ID NO: 9 - DNA, 37 bp, artificial sequence
  25bp 1 linker
ttttttttttttcgtggtcatgagccgggacgaagtc

SEQ ID NO: 10 - DNA, 37 bp, artificial sequence
  25bp 2 linker
aagagagagaaagacttcgtcccggctcatgaccacg

SEQ ID NO: 11 - DNA, 42 bp, artificial sequence
  30bp 1 linker
ttttttttttttcgtggtcatgagccgggacgaagtccaacc

SEQ ID NO: 12 - DNA, 42 bp, artificial sequence
  30bp 2 linker
aagagagagaaaggttggacttcgtcccggctcatgaccacg

SEQ ID NO: 13 - DNA, 40 bp, artificial sequence
  40bp 1 linker
gaggactatccggcgccgtccctcttcttcccttcttcct

SEQ ID NO: 14 - DNA, 40 bp, artificial sequence
  40bp 2 linker
gacggcgccggatagtcctcagcagagaataagaacaaag

SEQ ID NO: 15 - DNA, 50 bp, Artificial Sequence
  50bp 1 linker
tgggcgactcgacggcgccggatagtcctcagcagagaataagaacaaag

SEQ ID NO: 16 - DNA, 50 bp, artificial sequence
  50bp 2 linker
gaggactatccggcgccgtcgagtcgcccacctcttcttcccttcttcct

SEQ ID NO: 17 - DNA, 40 bp, artificial sequence
  60bp 1 linker
ttctttctttcctccttctccctcttcttcccttcttcct

SEQ ID NO: 18 - DNA, 40 bp, artificial sequence
  60bp 2 linker
gagaaggaggaaagaaagaaagcagagaataagaacaaag

SEQ ID NO: 19 - DNA, 32 bp, artificial sequence; identical to SEQ ID NO: 7
  70bp 1 linker

SEQ ID NO: 20 - DNA, 70 bp, artificial sequence
  70bp 2 linker
agccttactggtggtgccactgggcgactcgacggcgccggatagtcctcagcagagaataagaacaaag

SEQ ID NO: 21 - DNA, 80 bp, artificial sequence
  80bp 1 linker
gaggactatccggcgccgtcgagtcgcccagtggcaccaccagtaaggcttatcgcatgtcctcttcttcccttcttcct

SEQ ID NO: 22 - DNA, 80 bp, artificial sequence
  80bp 2 linker
acatgcgataagccttactggtggtgccactgggcgactcgacggcgccggatagtcctcagcagagaataagaacaaag

SEQ ID NO: 23 - DNA, 90 bp, Artificial Sequence
  90bp 1 linker
gaggactatccggcgccgtcgagtcgcccagtggcaccaccagtaaggcttatcgcatgtaagttgcacccctcttcttcccttcttcct

SEQ ID NO: 24 - DNA, 87 bp, artificial sequence
  90bp 2 linker
ggtgcaacttacatgcgataagccttactggtggtgccactgggcgactcgacggcgccggatagtcctcagcagagaataagaaca

SEQ ID NO: 25 - DNA, 100 bp, artificial sequence
  100bp 1 linker
gaggactatccggccccgtcgagtcgcccagtggcaccaccagtaaggcttatcgcatgtaagttgcacccccatcctcccctcttcttcccttcttcct

SEQ ID NO: 26 - DNA, 100 bp, artificial sequence
  100bp 2 linker
ggaggatgggggtgcaacttacatgcgataagccttactggtggtgccactgggcgactcgacggggccggatagtcctcagcagagaataagaacaaag

SEQ ID NO: 27 - protein, 247 aa, homo sapien
EQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYTYSDFNDGYYNYHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGLEVLF

SEQ ID NO: 28 - protein, 216 aa, Homo sapiens
QSALTQPASVSGSPGQTITISCQGTSSDVGGFDSVSWYQQSPGKAPKVMVFDVSHRPSGISNRFSGSKSGNTASLTISGLHIEDEGDYFCSSLTDRSHRIFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 29 - protein, 251 aa, homo sapiens
RLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNTYSTYSDFYDGYYNYHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGLEVLFQ

SEQ ID NO: 30 - protein, 216 aa, homo sapiens
QSALTQPASVSGSPGQSITISCQGTSNDVGGYESVSWYQQHPGKAPKVVIYDVSKRPSGVSNRFSGSKSGNTASLTISGLQAEDEGDYYCKSLTSTRRRVFGTGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 31 - protein, 229 aa, homo sapiens
QVHLSQSGAAVTKPGASVRVSCEASGYKISDHFIHWWRQAPGQGLQWVGWINPKTGQPNNPRQFQGRVSLTRQASWDFDTYSFYMDLKAVRSDDTAIYFCARQRSDFWDFDVWGSGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKT

SEQ ID NO: 32 - protein, 206 aa, homo sapiens
DIQMTQSPSSLSARVGDTVTITCQANGYLNWYQQRRGKAPKLLIYDGSKLERGVPARFSGRRWGQEYNLTINNLQPEDVATYFCQVYEFIVPGTRLDLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 230 aa, homo sapiens
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSC

SEQ ID NO: 34 - protein, 215 aa, homo sapiens
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 35 - protein, 236 aa, homo sapiens
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCD

SEQ ID NO: 36 - protein, 214 aa, homo sapiens
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 236 aa, homo sapiens
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCD

SEQ ID NO: 38 - protein, 211 aa, homo sapiens
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 236 aa, homo sapiens
EVQLVESGGGLVKPGGSLRLSCSASGFDFDNAWMTWVRQPPGKGLEWVGRITGPGEGWSVDYAAPVEGRFTISRLNSINFLYLEMNNLRMEDSGLYFCARTGKYYDFWSGYPPGEEYFQDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 40 - protein, 215 aa, homo sapiens
SYELTQETGVSVALGRTVTITCRGDSLRSHYASWYQKKPGQAPILLFYGKNNRPSGVPDRFSGSASGNRASLTISGAQAEDDAEYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 34 aa, artificial sequence
  TPR repeat
AXAWYNLGNAYYKQGDYDEAIXYYQKALELDPXX

SEQ ID NO: 42 - protein, 32 aa, artificial sequence
  Flexible Linker, Sortase A, BirA, His
GGGGASLPETGGLNDIFEAQKIEWHEHHHHHH

SEQ ID NO: 43 - protein, 29 aa, artificial sequence
  GlySer Linker Sortase A, His
ASGGGGSGGGGSGGGGSLPETGGHHHHHH

(19) United States
(12) Patent Application Publication
(10) Pub. No.: US 2016/0102137 A1
(43) Pub. Date: Apr. 14, 2016

(54) CROSSLINKED ANTI-HIV-1 COMPOSITIONS FOR POTENT AND BROAD NEUTRALIZATION

(71) Applicant: CALIFORNIA INSTITUTE OF TECHNOLOGY, Pasadena, CA (US)

(72) Inventors: Pamela J. Bjorkman, Altadena, CA (US); Rachel P. Galimidi, Pasadena, CA (US); Anthony P. West, Pasadena, CA (US); Michel C. Nussenzweig, New York, NY (US)

(21) Appl. No.: 14/872,051

(22) Filed: Sep. 30, 2015

Related U.S. Application Data
(60) Provisional application No. 62/057,405, filed on Sep. 30, 2014.

(51) Int. Cl. C07K 16/10; C12N 15/113; C07K 16/46

(52) U.S. Cl. CPC C07K 16/1063; C07K 16/468; C12N 15/1132; C07K 2317/55; C07K 2317/31; C07K 2319/00; C12N 2310/11; C12N 2310/3513; C12N 2320/30

(57) ABSTRACT

An anti-HIV-1 spike composition includes a first anti-HIV-1 antibody Fab and a second anti-HIV-1 antibody Fab linked by a DNA or protein linker molecule to form a crosslinked homo-diFab or hetero-diFab having improved viral potency and neutralization. The anti-HIV-1 antibody Fabs include anti-gp120 CD4, anti-gp120 V1V2, anti-gp120 V3, and anti-gp41.

### CROSS-REFERENCE TO RELATED APPLICATION(S)

The present application claims priority to and the benefit of U.S. Provisional Application Ser. No. 62/057,405 filed on Sep. 30, 2014, the entire contents of which is incorporated herein by reference.

### STATEMENT REGARDING FEDERALLY SPONSORED RESEARCH OR DEVELOPMENT

This invention was made with government support under Grant No. OD006961 awarded by the National Institutes of Health. The government has certain rights in the invention.

### BACKGROUND

Antibodies developed during human immunodeficiency virus-1 (HIV-1) infection lose efficacy as the viral spike mutates. It is thought that anti-HIV-1 antibodies primarily bind monovalently because HIV's low spike density impedes bivalent binding through inter-spike crosslinking, and the spike structure prohibits bivalent binding through intra-spike crosslinking. Monovalent binding reduces avidity and potency, thus expanding the range of mutations permitting antibody evasion.

The HIV-1 envelope (Env) spike trimer, a trimer complex of gp120 and gp41 subunits, is the only target of neutralizing antibodies. The spike utilizes antibody-evasion strategies including mutation, glycan shielding, and conformational masking. An antibody-evasion strategy that is possibly unique to HIV-1 involves hindering IgGs from using both antigen-binding fragments (Fabs) to bind bivalently to spikes. This is accomplished by the small number and low density of Env spikes, which prevent most IgGs from inter-spike crosslinking (bivalent binding between spikes), and the architecture of the Env trimer, which impedes intra-spike crosslinking (bivalent binding within a spike trimer).

On a typical virus with closely-spaced envelope spikes, an IgG antibody can bind using both Fabs to crosslink neighboring spikes, leading to a nearly irreversible antibody-antigen interaction. The small number of spikes (approximately 14) present on the surface of HIV-1 impedes simultaneous engagement of both antibody combining sites because most spikes are separated by distances that far exceed the approximate 15 nm reach of the two Fab arms of an IgG (FIG. 1). Accordingly, the mechanisms to hinder inter- and intra-spike crosslinking demonstrate that most anti-HIV-1 IgGs bind monovalently to virions.

### SUMMARY

In some embodiments of the present invention, an anti-HIV-1 composition includes a first anti-HIV-1 antibody Fab, a second anti-HIV-1 antibody Fab, and a linker molecule conjugated to the first anti-HIV-1 antibody Fab and the second anti-HIV-1 antibody Fab.

In some embodiments of the present invention, the linker molecule is selected from single stranded nucleic acids, double stranded nucleic acids, amino acids, proteins, or combinations thereof.

In some embodiments of the present invention, the first anti-HIV-1 antibody Fab and the second anti-HIV-1 antibody Fab are each independently selected from anti-gp120 V1V2 Fabs, anti-gp120 V3 Fabs, anti-gp120 CD4 Fabs, and/or anti-gp41 Fabs.

In some embodiments of the present invention, the linker molecule includes a first nucleic acid including a first segment conjugated at its 5′ end to the first anti-HIV antibody Fab and conjugated at its 3′ end to a sense strand of DNA, and a second nucleic acid including a second segment conjugated at its 5′ end to the second anti-HIV antibody Fab and conjugated at its 3′ end to an anti-sense strand of DNA complementary to the sense strand of DNA of the first nucleic acid.

In some embodiments of the present invention, the sense strand of DNA and the anti-sense strand of DNA each have a length selected from 20 to 100 base pairs, 25 to 80 base pairs, 30 to 70 base pairs, or 40 to 60 base pairs.

In some embodiments of the present invention, the linker molecule comprises from 3 tetratricopeptide repeat (TPR)(SEQ ID NO: 41) domains up to 30 TPR domains.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic of immunoglobulin (IgG) binding monovalently to spikes on HIV-1 surfaces, which include a small number (approximately 14) and low density of Env protein complex.

FIG. 2 is a schematic showing monovalently binding of anti-HIV-1 spike Fab, IgG, and crosslinked homo-diFabs and hetero-diFabs, according to embodiments of the present invention.

FIG. 3 is a schematic showing crosslinking of anti-HIV-1 spike Fabs using a double stranded DNA (dsDNA) linker molecule and a protein linker molecule, according to embodiments of the present invention.

FIG. 4 is a schematic of a method for crosslinking two Fab proteins by chemically modifying the C-terminus of each Fab and conjugating single stranded DNA (ssDNA) to the modified Fabs, followed by ligation of dsDNA to form the conjugated diFab with dsDNA linker molecule, according to embodiments of the present invention.

FIG. 5 is a schematic depicting a method Steps 1-4 for making homo- and hetero-diFabs. Step 1: Mild reduction of Fab containing a free thiol group at C-terminus of the heavy chain. Step 2: An amine-modified ssDNA oligonucleotide is reacted with Sulfo-SMCC (amine-to-sulfhydryl crosslinker) to form a maleimide-activated ssDNA. Step 3: The reduced Fab and maleimide-activated ssDNA are incubated to form a Fab conjugated to ssDNA. Step 4: Two ssDNA-conjugated Fabs (identical Fabs for making homo-diFabs; different Fabs for making hetero-diFabs) are joined with a dsDNA containing overhangs complementary to the ssDNA, and then ligated to form a homo- or hetero-diFab, according to embodiments of the present invention.

FIG. 6 shows size exclusion chromatography profiles for hetero-diFabs. Examples from which PG16-60 bp-b12 (left) and 3BNC60-60 bp-b12 (right) hetero-diFabs were isolated are shown (solid red line: A₂₆₀; solid blue line: A₂₈₀). The migration of a Fab that was not linked to DNA is shown for comparison (dashed red line: A₂₆₀; dashed blue line: A₂₈₀), according to embodiments of the present invention.

FIG. 7 shows SDS-PAGE analysis for PG16-60 bp-b12 purification. Size exclusion chromatography fractions were assayed by 10% SDS-PAGE (stained with Coomassie Blue for protein or with ethidium bromide for DNA), according to embodiments of the present invention.

FIG. 8 shows dynamic light scattering measurements of hydrodynamic radii for IgG and Fab proteins, different lengths of dsDNA alone, and di-Fabs with different dsDNA linkers, according to embodiments of the present invention.

FIG. 9 shows graphical analysis of the effects of dsDNA bridge length on neutralization potencies of 3BNC60 and PG16 homo-diFabs against the Tier 1B HIV-1 strain 6535.3. Neutralization IC₅₀s are plotted against the length of the dsDNA linker. IC₅₀s for the parent IgG and Fab are indicated as red and blue lines, respectively, according to embodiments of the present invention.

FIG. 10 is a table showing neutralization data of primary HIV-1 strains by b12 and PG16 homo-diFabs, each constructed with a 60 bp dsDNA bridge. IC₅₀s are reported for the homo-diFabs, the parental Fabs and IgGs, and dsDNA alone. As a measure of potential synergy, the molar ratio of the IC₅₀ values for the IgG and the homo-diFab is listed for each strain in parentheses beside the IC₅₀ for the homo-diFab, according to embodiments of the present invention.

FIGS. 11A-11D show IC₅₀s for neutralization by homo-diFabs of the indicated HIV-1 strains plotted against the length of the dsDNA linker. In each plot, the Fab in the homo-diFab is listed before the viral strain against which the reagents were evaluated. IC₅₀s for the analogous IgG and Fab are indicated as red (IgG) and blue (Fab) lines. NT (not tested) indicates an IC₅₀ that was not derived. FIG. 11A shows anti-CD4bs homo-diFabs 3BNC60 and VRC01), FIG. 11B shows b12 (anti-CD4) homo-diFab, FIG. 11C shows 10-1074 (anti-gp120 V3) homo-diFab, and FIG. 11D shows PG16 (anti-gp120V1V2) homo-diFab, according to embodiments of the present invention.

FIG. 12 shows three conformations of Env trimers shown as surface representations (top row: gp120 coordinates only) and schematically (bottom two rows). Schematic representations of Env trimers. Env spikes are shown as seen from above (top and middle rows) and the side (bottom row). V1V2 loops are cyan, V3 loops are purple, the CD4 binding site is yellow, the remainder of gp120 is maroon, gp41 is green, and the membrane bilayer is gray. The closed structure (PDB code 4NCO) was observed for unliganded trimers and trimers associated with Fabs from potent VRC01-like (PVL) antibodies. The open structure was observed for trimers associated with CD4 or the Fab from the CD4-induced antibody 17b (coordinates obtained from S. Subramaniam). The partially-open structure was observed for trimers associated with the Fab from b12 (PDB code 3DNL).

FIG. 13 schematically depicts measured distances between homo-diFabs bound to HIV-1 trimer structures. Fabs from the indicated bNAbs shown bound to the gp120 portions of Env in the three conformations shown in panel A. Fabs are shown as ribbons; gp120 subunits are shown as surface representations with V1V2 loops in cyan, V3 in purple, the CD4 binding site in yellow, and the remainder of gp120 in maroon. The distance between the Cys233_{heavy} chain carbon-□ atoms of adjacent bound Fabs is indicated by a gray line as an approximation of an optimal length for a dsDNA bridge attached to Cys233_{heavy chain}. Assuming three-fold symmetry of trimers, only one distance is possible for bound 3BNC60, b12 and 10-1074 homo-diFabs.

FIG. 14 Fabs from the indicated bNAbs shown bound to the gp120 portions of Env in three conformations: closed, partially open, and open. Fabs are shown as ribbons; gp120 subunits are shown as surface representations with V1V2 loops in cyan, V3 in purple, the CD4 binding site in yellow, and the remainder of gp120 in maroon. The distance between the Cys233_{heavy chain} carbon-α atoms of adjacent bound Fabs is indicated by a gray line as an approximation of an optimal length for a dsDNA bridge attached to Cys233_{heavy chain}. Three distances are possible for hetero-diFabs binding to Env trimer. The distance between Fabs bound to the same gp120 subunit (thick line) remains the same in the three trimer conformations.

FIG. 15 is a table showing the neutralization data of primary HIV-1 strains by hetero-diFabs, as indicated, according to embodiments of the present invention. IC₅₀s are reported for the hetero-diFabs. As a measure of potential synergy of each hetero-diFab, the molar ratio of the IC₅₀ values for the non-covalent mixture and the hetero-diFab is listed for each strain in parentheses beside the IC₅₀ for the hetero-diFab. NT=not tested.

FIG. 16 is a table showing the neutralization data of primary HIV-1 strains by hetero-diFabs, as indicated, according to embodiments of the present invention. IC₅₀s are reported for the hetero-diFabs. As a measure of potential synergy of each hetero-diFab, the molar ratio of the IC₅₀ values for the non-covalent mixture and the hetero-diFab is listed for each strain in parentheses beside the IC₅₀ for the hetero-diFab. NT=not tested.

FIG. 17 is a table showing the IC₅₀ values for neutralization of primary HIV-1 strains by PG16-60 bp-b12 hetero-diFab, according to embodiments of the present invention. IC₅₀s are reported for the hetero-diFab, the parental Fabs and IgGs, the dsDNA bridge alone, and a non-covalent mixture of the Fabs and the dsDNA bridge. As a measure of potential synergy of the hetero-diFab, the molar ratio of the IC₅₀ values for the non-covalent mixture and the hetero-diFab is listed for each strain in parentheses beside the IC₅₀ for the hetero-diFab.

FIG. 18 is a table showing the IC₅₀ values for neutralization of primary HIV-1 strains by PG16-3BNC60 hetero-diFabs, according to embodiments of the present invention. IC₅₀s are reported for the hetero-diFab, the parental Fabs and IgGs, the dsDNA bridge alone, and a non-covalent mixture of the Fabs and the dsDNA bridge. As a measure of potential synergy of the hetero-diFab, the molar ratio of the IC₅₀ values for the non-covalent mixture and the hetero-diFab is listed for each strain in parentheses beside the IC₅₀ for the hetero-diFab.

FIG. 19 is a table showing the IC₅₀ values for neutralization of primary HIV-1 strains by PG9-60 bp-3BNC60 hetero-diFab, according to embodiments of the present invention. IC₅₀s are reported for the hetero-diFab, the parental Fabs and IgGs, the dsDNA bridge alone, and a non-covalent mixture of the Fabs and the dsDNA bridge. As a measure of potential synergy of the hetero-diFab, the molar ratio of the IC₅₀ values for the non-covalent mixture and the hetero-diFab is listed for each strain in parentheses beside the IC₅₀ for the hetero-diFab.

FIG. 20 is a table showing the IC₅₀ values for neutralization of primary HIV-1 strains by 10-1074-3BNC60 and 10E8-3BNC60 heterodi-Fabs, according to embodiments of the present invention. IC₅₀s are reported for the hetero-diFab, the parental Fabs and IgGs, the dsDNA bridge alone, and a non-covalent mixture of the Fabs and the dsDNA bridge. As a measure of potential synergy of the hetero-diFab, the molar ratio of the IC₅₀ values for the non-covalent mixture and the hetero-diFab is listed for each strain in parentheses beside the IC₅₀ for the hetero-diFab.

FIG. 21 is a table showing the IC₅₀ values for neutralization of primary HIV-1 strains by 3BNC60-60 bp-b12 hetero-diFab, according to embodiments of the present invention. IC₅₀s are reported for the hetero-diFab, the parental Fabs and IgGs, the dsDNA bridge alone, and a non-covalent mixture of the Fabs and the dsDNA bridge. As a measure of potential synergy of the hetero-diFab, the molar ratio of the IC₅₀ values for the non-covalent mixture and the hetero-diFab is listed for each strain in parentheses beside the IC₅₀ for the hetero-diFab.

FIG. 22 are graphs of the amount of neutralization of the indicated viral strains compared for hetero-diFabs (separated by different dsDNA bridge lengths), each of the parent Fabs alone, a non-covalent mixture of the parent Fabs plus dsDNA, and (when available) the analogous heterodimeric IgG, with the upper panels showing. PG16-60 bp-b12 hetero-diFab and controls as indicated and the lower panels showing PG16-3BNC60 hetero-diFabs and controls, as indicated, according to embodiments of the present invention. IC₅₀ values are shown on the right. Error bars represent standard deviations of measurements at each concentration.

FIG. 23 are graphs of the amount of neutralization of the indicated viral strains compared for hetero-diFabs (separated by different dsDNA bridge lengths), each of the parent Fabs alone, a non-covalent mixture of the parent Fabs plus dsDNA, and (when available) the analogous heterodimeric IgG, with the upper panels showing. 10-1074-3BNC60 hetero-diFabs and controls as indicated and the lower panels showing 10E8-3BNC60 hetero-diFabs and controls, as indicated, according to embodiments of the present invention. IC₅₀ values are shown on the right. Error bars represent standard deviations of measurements at each concentration.

FIG. 24 is a schematic representation of the conjugation of the protein linked hetero-diFab PG16-TPR12-3BNC60 (not to scale), according to embodiments of the present invention, with the approximate lengths indicated (120 Å for the TRP12 protein linker plus approximately 11 Å for the fused click handles.

FIG. 25 is a table showing the neutralization data of primary HIV-1 strains with PG16-TPR12-3BNC60, according to embodiments of the present invention. IC₅₀s are reported for PG16-TPR12-3BNC60, the parental components of the reagent (PG16 Fab and 3BNC60 Fab-TPR12), and TPR12 alone. As a measure of potential synergy of PG16-TPR12-3BNC60, the molar ratio of the IC₅₀ values for the most potent component and PG16-TPR12-3BNC60 is listed for each strain in parentheses beside the IC₅₀ for PG16-TPR12-3BNC60.

FIG. 26 shows the Size exclusion chromatography (SEC) profiles for PG16-TPR12-3BNC60, according to embodiments of the present invention; SEC runs from which PG16-TPR12-3BNC60 was isolated from fractions 10.3 mL-11.8 mL. SEC profiles are shown for 3BNC60 Fab-TPR12 and PG16 Fab for comparison.

FIG. 27 shows simulations of avidity effects due to bivalent binding of IgG to a tethered antigen, according to embodiments of the present invention. The fraction of tethered antigen bound by different concentrations of IgG or Fab after 1 hour are shown as a heat map (cooler colors representing a lower percentage bound and warmer colors representing a higher percentage bound) as a function of kinetic constants for the IgG-antigen or Fab-antigen interaction. The fraction of antigen bound by a Fab or IgG was calculated as a function of kₐ and k_{d}. The intrinsic affinities are strongest in the lower right corner (1 pM) and weakest in the upper left corner (100 mM) of each graph. For IgG, binding was forced to 100% monovalent binding (middle row) or 100% bivalent binding (bottom row). Saturation by Fabs and IgGs was nearly identical for monovalent binding conditions because the binding kinetics of IgGs would be enhanced by at most 2-fold. Comparisons of the simulations for bivalent binding (bottom row) and monovalent binding (top two panels) showed regions of saturation binding resulting from avidity effects.

FIG. 28 are graphs showing the fraction of antigen bound as a function of time for IgGs binding to surface-tethered antigens at an input concentration of 10 nM, according to embodiments of the present invention. When the dissociation rate constant of the Fab portion of the IgG is slow (top panel) and the input concentration is approximately 100-fold higher than the affinity of the Fab, IgGs can reach saturation binding after an hour whether binding monovalently or bivalently to the surface—hence avidity effects are not apparent after an hour. However, weakening the affinity of the Fab by making the dissociation rate 1000-fold faster (bottom panel) prevents saturation when binding monovalently, but has no effect on saturation when binding bivalently—hence avidity effects are apparent throughout the incubation.

FIG. 29 is a schematic representation of the utility of the dsDNA linker molecules for rendering bivalent crosslinked anti-HIV-1 diFabs as disclosed herein, according to embodiments of the present invention.

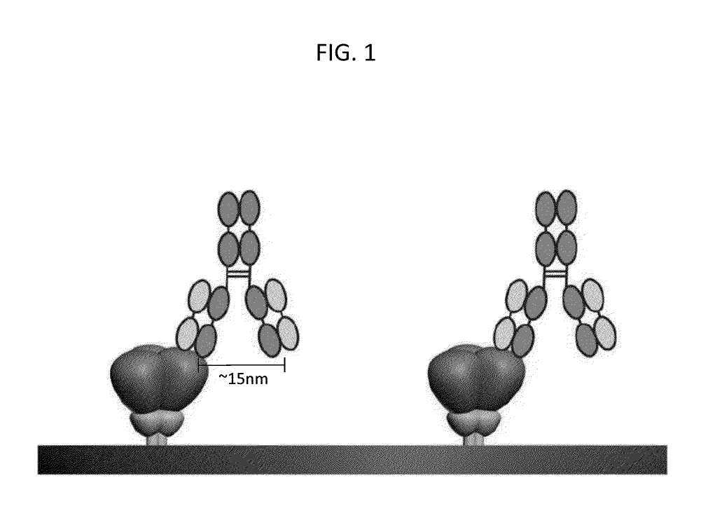

### DETAILED DESCRIPTION

Engineered anti-HIV-1 spike-binding Fab molecules designed to bind bivalently demonstrate that avidity effects correlate with antibody efficacy in HIV-1 neutralization. As described in the present disclosure, engineered anti-HIV-1 spike antibody Fabs that bind to HIV-1 envelope (Env) proteins are modified by linker molecules to conjugate two Fab molecules together, resulting in bivalent binding to the HIV-1 spike complex and increased viral neutralization.

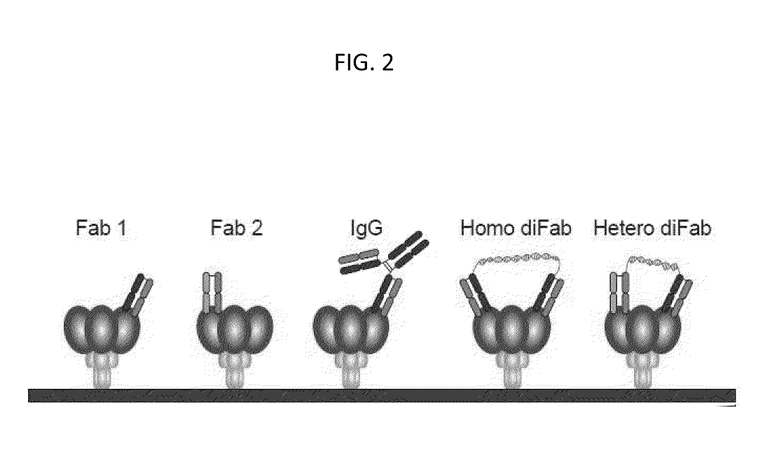

In some embodiments of the present disclosure, a crosslinked bivalent binding composition for anti-HIV-1 includes two anti-HIV spike antibody Fabs that have the same antigen binding residues resulting in a crosslinked homo-diFab, as shown in FIG. 2. In some embodiments, a crosslinked bivalent binding composition for anti-HIV-1 includes two anti-HIV spike antibody Fabs that have different antigen binding residues resulting in a crosslinked hetero-diFab, as shown in FIG. 2 in which the hetero-diFab is binding the gp120 protein and the gp41 protein of the spike complex.

As used herein, the term “homo-diFab” and like terms refer to two crosslinked Fab (antibody binding fragment) proteins that have the same antigen binding interface, and therefore the same residues on each of the Fab proteins bind to the antigen. As such, homo-diFabs may have two identical Fab proteins having the same amino acid sequence and structure throughout. Homo-diFabs may also have two Fab proteins that have the same antigen binding residues, but that have differing protein sequences throughout the rest of the respective Fab proteins.

As used herein, the term “hetero-diFab” and like terms refer to two crosslinked Fab proteins having different antigen binding residues. Hetero-diFabs may include two Fabs that bind the same HIV-1 protein (e.g., gp120) but at different antigenic sites within that protein (e.g., CD4 and V1V2), as schematically shown in FIG. 2.

As used herein, with respect to a Fab or immunoglobulin (IgG) protein, “binding residues,” “interface,” “binding interface, “binding interface residues,” and like terms refer to the amino acid residues of the Fab or IgG protein that bind directly to an epitope on an HIV-1 protein.

As used herein, an antibody Fab or IgG that binds gp120 at the residues of gp120 that bind to the CD4 protein, may be referred to as an anti-gp120 CD4 Fab, anti-gp120 CD4 IgG, or anti-gp120 CD4, and the like.

As used herein, an antibody Fab or IgG that binds the variable regions 1 and 2 (V1/V2) of gp120 may be referred to as an anti-gp120 V1V2 Fab, anti-gp120 V1V2 IgG, anti-gp120 V1V2, and the like.

As used herein, an antibody Fab or IgG that binds the third variable loop region (V3) of gp120 may be referred to as an anti-gp120 V3 Fab, anti-gp120 V3 IgG, anti-gp120 V3, and the like.

As used herein, an antibody Fab or IgG that binds gp41 is referred to as an anti-gp41 Fab, anti-gp41 IgG, anti-gp41, and the like.

As used herein, “conjugated,” “conjugation” and like terms refer to the linkage between and amongst nucleic acids, amino acids of peptide and/or proteins, chemical moieties, and combinations of each of these as described in this disclosure for connecting the two anti-HIV-1 Fabs with a linker molecule. Conjugation includes the covalent bonding between two amino acids, the covalent bonding between nucleotides in a single chain of nucleic acids, the covalent bonding between a nucleotide and an amino acid, the covalent bonding between a chemical moiety (e.g., azide or cyclooctyne) and an amino acid, and the covalent bonding between a chemical moiety and a nucleotide.

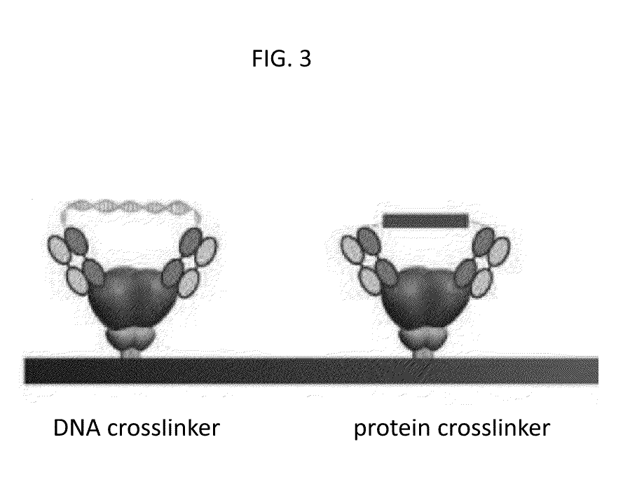

As used herein, “linker,” “linker molecule,” “crosslinker,” “crosslinker molecule,” and like terms refer to the molecule that conjugates to the C-terminus of each of two anti-HIV-1 antibody Fabs. The linker molecule may be a heteromolecule that includes more than one type of molecule such as chemical moieties, single stranded nucleic acids, double stranded nucleic acids, (e.g., DNA), amino acids, peptides, and/or proteins. Both a DNA crosslinker and a protein crosslinker are schematically depicted in FIG. 3.

As used herein, “segment” and like terms refer to a part, a domain, or a region of the linker molecule made of one type of molecule. A segment may be contiguous with another type of molecule forming a larger heteromolecule.

Abbreviations for amino acids are used throughout this disclosure and follow the standard nomenclature known in the art. For example, as would be understood by those of ordinary skill in the art, Alanine is Ala or A; Arginine is Arg or R; Asparagine is Asn or N; Aspartic Acid is Asp or D; Cysteine is Cys or C; Glutamic acid is Glu or E; Glutamine is Gln or Q; Glycine is Gly or G; Histidine is His or H; Isoleucine is Ile or I; Leucine is Leu or L; Lysine is Lys or K; Methionine is Met or M; Phenylalanine is Phe or F; Proline is Pro or P; Serine is Ser or S; Threonine is Thr or T; Tryptophan is Trp or W; Tyrosine is Tyr or Y; and Valine is Val or V.

An antibody or antibody Fab of the present invention may be a “humanized antibody” or “humanized Fab”. A humanized antibody Fab is considered to be a human Fab that has one or more amino acid residues introduced into it from a source that is non-human. These non-human amino acid residues often are referred to as “import” residues, which typically are taken from an “import” variable region. Humanization may be performed following known methods by substituting import hypervariable region sequences for the corresponding sequences of a human antibody. (See, for example, Jones et al., Nature, 321:522-525 20 (1986); Reichmann et al., Nature, 332:323-327 (1988); Verhoeyen et al., Science, 239:1534-1536 (1988)) the entire contents of each are incorporate herein by reference). Accordingly, such “humanized” antibodies are chimeric antibodies in which substantially less than an intact human variable region has been substituted by the corresponding sequence from a non-human species.

### Anti-HIV-1 Antibody Fabs

In some embodiments, anti-HIV-1 antibody Fabs (also referred herein as Fab protein, anti-HIV-1 antibody Fab proteins, and the like) include anti-gp120 V1V2 Fab, anti-gp120 CD4 Fab, anti-gp120 V3, and anti-gp41. In some embodiments of the present invention, as disclosed in the Examples, the Fab proteins may be modified for conjugation to a linker molecule. For example, the cysteine (Cys263) residue on the Fab light chain may be modified by site-directed mutagenesis to preclude the formation of a disulfide bond with Cys233 of the Fab heavy chain.

Anti-gp120 V1V2 Fab.

In some embodiments of the present invention, the anti-gp120 V1V2 Fab has a Fab heavy chain and a Fab light chain in which the heavy chain includes binding interface residues corresponding to positions 57-59, 61, 64, 100, 100B, 100D, 100E, 100F, 100G, 100H, 100I, 100J, 100K, 100L, 100O, 100P, 100Q, and 100R based on PDB 4DQO, and the light chain includes binding interface residues corresponding to positions 31, 32, 50, 91, 94, and 95A based on PDB 4DQO.

In some embodiments of the present invention, the anti-gp120 V1V2 Fab has heavy chain binding interface residues corresponding to LYS57, TYR58, HIS59, ASP61, TRP64, ILE100, HIS100B, ASP100D, VAL100E, LYS100F, TYR100G, TYR100H, ASP100I, PHE100J, ASN100K, ASP100L, TYR100O, ASN100P, TYR100Q, and HIS100R, and light chain binding interface residues corresponding to ASP31, SER32, ASP50, LEU91, ARG94, and HIS95A based on PDB 4DQO.

In some embodiments of the present invention, the anti-gp120 V1V2 Fab corresponds to PDB 4DQO for PG16 (heavy chain: SEQ ID NO: 27, light chain: SEQ ID NO: 28) with C-terminal modifications as disclosed herein.

In other embodiments of the present invention, the anti-gp120 V1V2 Fab has a Fab heavy chain and a Fab light chain in which the heavy chain includes binding interface residues corresponding to positions 31, 53, 55, 100, 100B, 100E, 100F, 100G, 100H, 100I, 100J, 100K, 100L, 100O, 100P, 100Q, and 100R and the light chain includes binding interface residues corresponding to positions 31, 32, 50, 91, 94, and 95A based on PDB 3U2S.

In still other embodiments of the present invention, the anti-gp120 V1V2 Fab has heavy chain binding interface residues corresponding to ARG31, ASP53, SER55, ASP100, ARG100B, TYR100E, ASN100F, TYR100G, TYR100H, ASP100I, PHE100J, TYR100K, ASP100L, TYR100O, ASN100P, TYR100Q, and HIS100R and the light chain includes binding interface residues corresponding to GLU31, SER32, ASP50, and LEU91, based on PDB 3U2S.

In some embodiments of the present invention, the anti-gp120 V1V2 Fab corresponds to PDB 3U2S for PG9 (heavy chain: SEQ ID NO: 29, light chain: SEQ ID NO: 30) with C-terminal modifications as disclosed herein.

Anti-gp120 CD4 Fab.

In some embodiments of the present invention, the anti-gp120 CD4 Fab has a Fab heavy chain and a Fab light chain in which the heavy chain includes binding interface residues corresponding to positions 30, 47, 50, 53-58, 60, 61, 64, 71, 71D, 72, 98, and 100, and the light chain includes binding interface residues corresponding to positions 27, 32, 91, 96, and 97 based on PDB 4JPV for 3BNC117 (3BNC117 shares the same interface binding residues with 3BNC60).

In some embodiments of the present invention, the anti-gp120 CD4 Fab has heavy chain binding interface residues corresponding to SER30, TRP47, TRP50, LYS53, THR54, GLY55, GLN56, PRO57, ASN58, PRO60, ARG61, GLN64, ARG71, TRP71D, ASP72, ASP98, and TRP100, and the light chain includes binding interface residues corresponding to GLY27, TYR32, TYR91, GLU96, and PHE97, based on PDB 4JPV for Fab 3BNC117 (3BNC117 shares the same interface binding residues with 3BNC60).

In some embodiments of the present invention, the anti-gp120 CD4 Fab corresponds to PDB 3RPI for 3BNC60 (heavy chain: SEQ ID NO: 31, light chain: SEQ ID NO: 32) with C-terminal modifications as disclosed herein.

In other embodiments of the present invention, the anti-gp120 CD4 Fab has a Fab heavy chain and a Fab light chain in which the heavy chain includes binding interface residues corresponding to positions 28, 30-33, 52-54, 56, 96-100, 100G, and 100H, based on PDB 2NY7.

In still other embodiments of the present invention, the anti-gp120 CD4 Fab has heavy chain binding interface residues corresponding to ARG28, SER30, ASN31, PHE32, VAL33, ASN52, TYR53, ASN54, ASN56, GLY96, PRO97, TYR98, SER99, TRP100, ASN100G, TYR100H, based on PDB 2NY7.

In some embodiments of the present invention, the anti-gp120 CD4 Fab corresponds to PDB 2NY7 for b12 (heavy chain: SEQ ID NO: 33, light chain: SEQ ID NO: 34) with C-terminal modifications as disclosed herein.

Anti-gp120 V3 Fab.

In some embodiments of the present invention, the anti-gp120 V3 Fab corresponds to PDB 4FQ2 for 10-1074 (heavy chain: SEQ ID NO: 35, light chain: SEQ ID NO: 36) with C-terminal modifications as disclosed herein.

In other embodiments of the present invention, the anti-gp120 V3 Fab corresponds to PDB 4FQ1 for PGT121 (heavy chain: SEQ ID NO: 37, light chain: SEQ ID NO: 38) with C-terminal modifications as disclosed herein.

Anti-gp41 Fab.

In some embodiments of the present invention, the anti-gp41 Fab has a Fab heavy chain and a Fab light chain in which the heavy chain includes binding interface residues corresponding to positions 28, 31, 33, 52, 52B, 52C, 53, 56, 97-99, 100A, 100B, 100D, 100E, 100F, and 100G, and the light chain includes a binding interface residue corresponding to position 95B, based on PDB 4G6F.

In some embodiments of the present invention, the anti-gp41 Fab has heavy chain binding interface residues corresponding to ASP28, ASN31, TRP33, THR52, PRO52B, GLY52C, GLU53, SER56, LYS97, TYR98, TYR99, PHE100A, TRP100B, GLY100D, TYR100E, PRO100F, PRO100G, and the light chain includes a binding interface residue corresponding to ARG95B, based on PDB 4G6F.

In some embodiments of the present invention, the anti-gp41 Fab corresponds to PDB 4G6F for 10E8 (heavy chain: SEQ ID NO: 39, light chain: SEQ ID NO: 40) with C-terminal modifications as disclosed herein.

Anti-HIV-1 diFabs Crosslinked with Double Stranded DNA

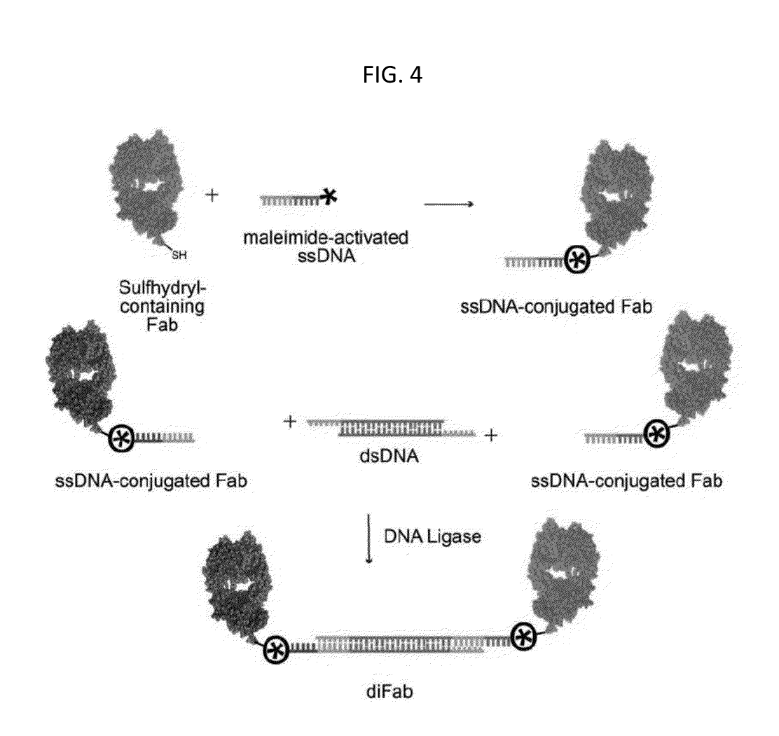

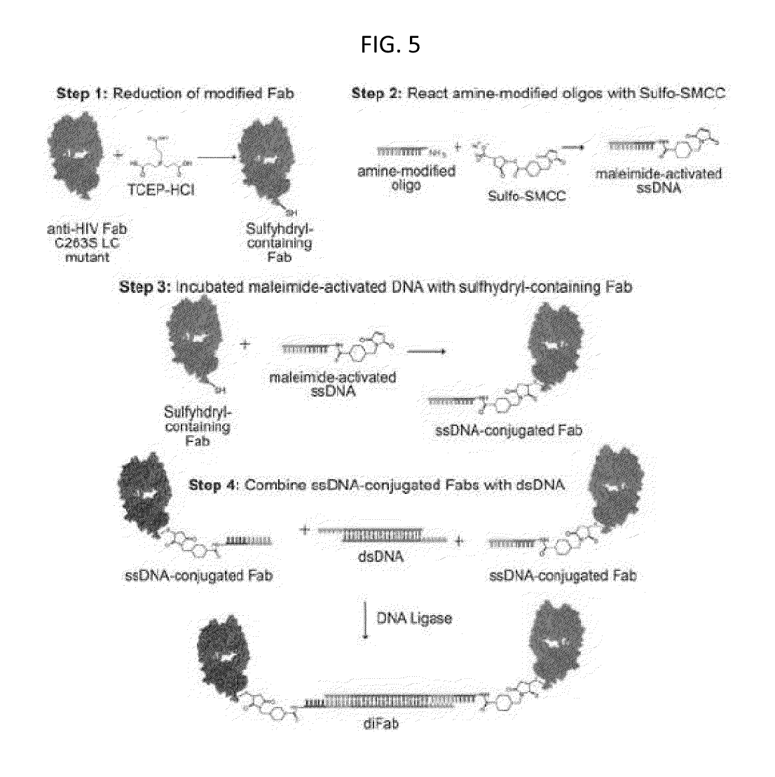

In order to establish effective crosslinker lengths between various anti-HIV-1 Fab antibodies, Fab proteins were modified and conjugated to linker molecules made of single stranded nucleic acid linkers and double stranded nucleic acid bridges (e.g., the bridges having paired sense and anti-sense strands of DNA), as shown in FIGS. 4 and 5, and described in more detail in this disclosure.

Table 1 (Example 6) shows a list of varying length sequences (SEQ ID Nos. 1-26) used to establish desired ranges for combinations of anti-HIV-1 spike Fabs. Using dsDNA linkers from Table 1 with anti-HIV-1 spike Fabs, diFabs were analyzed using viral neutralization assays.

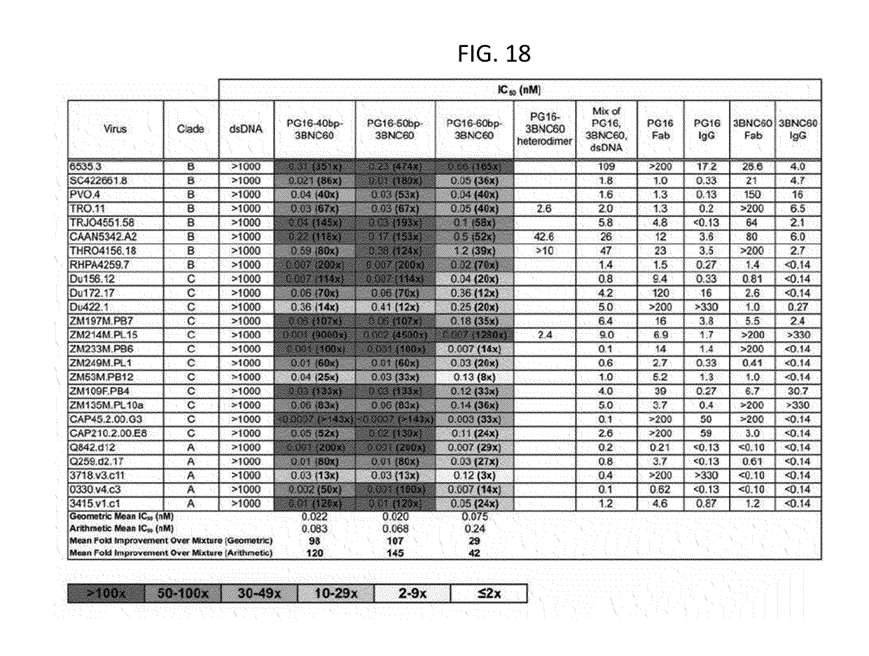

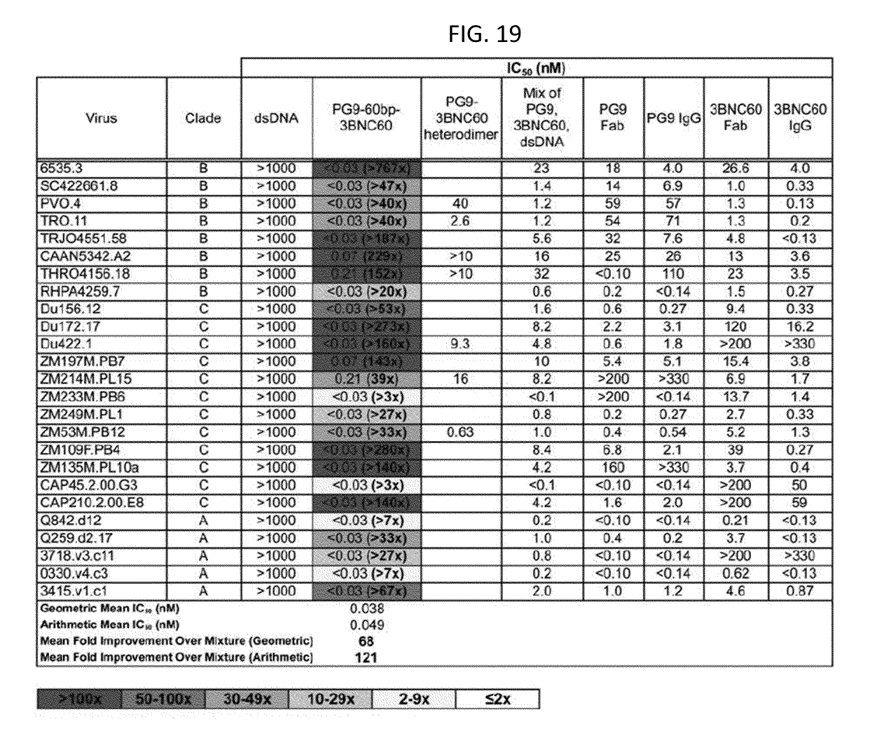

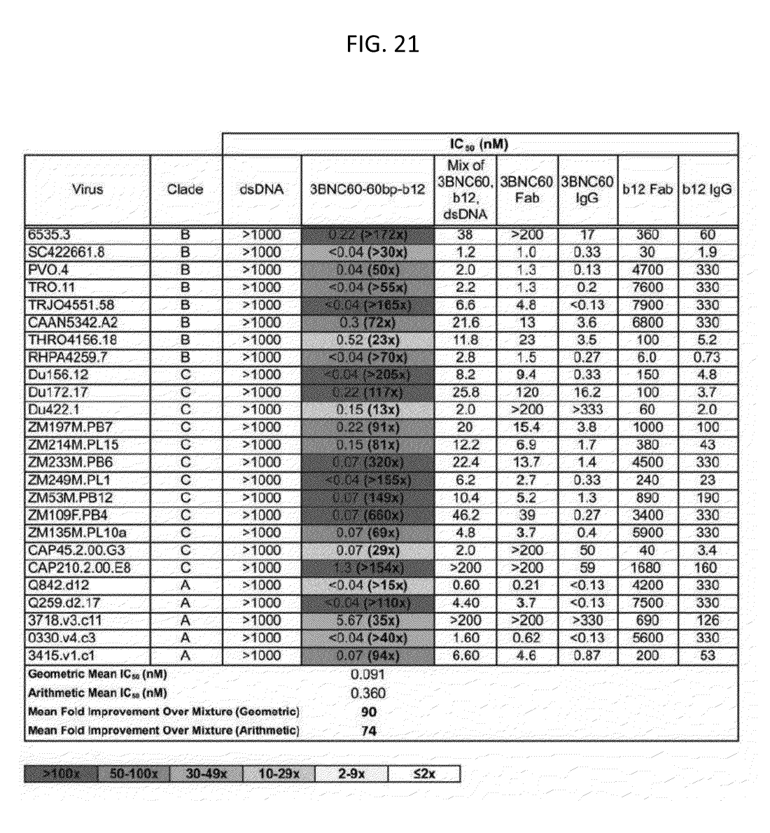

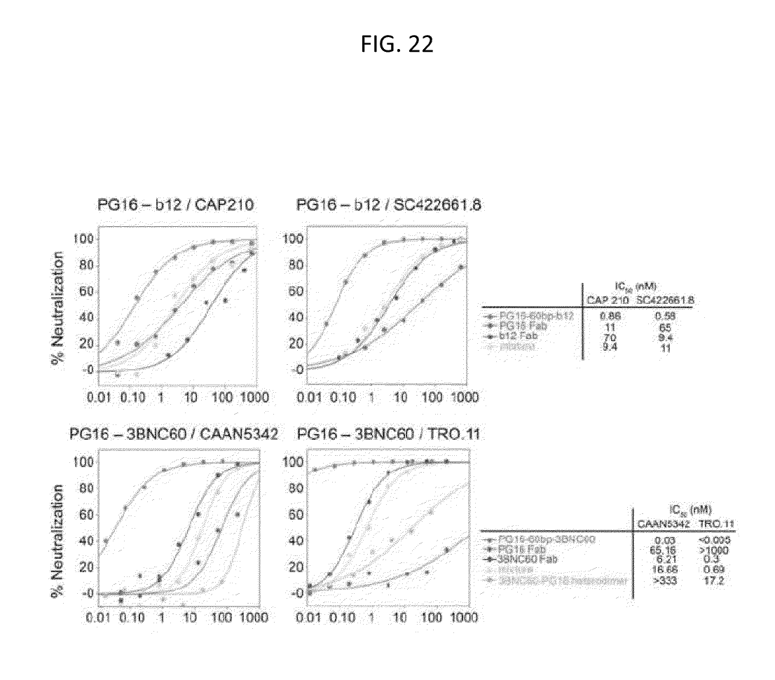

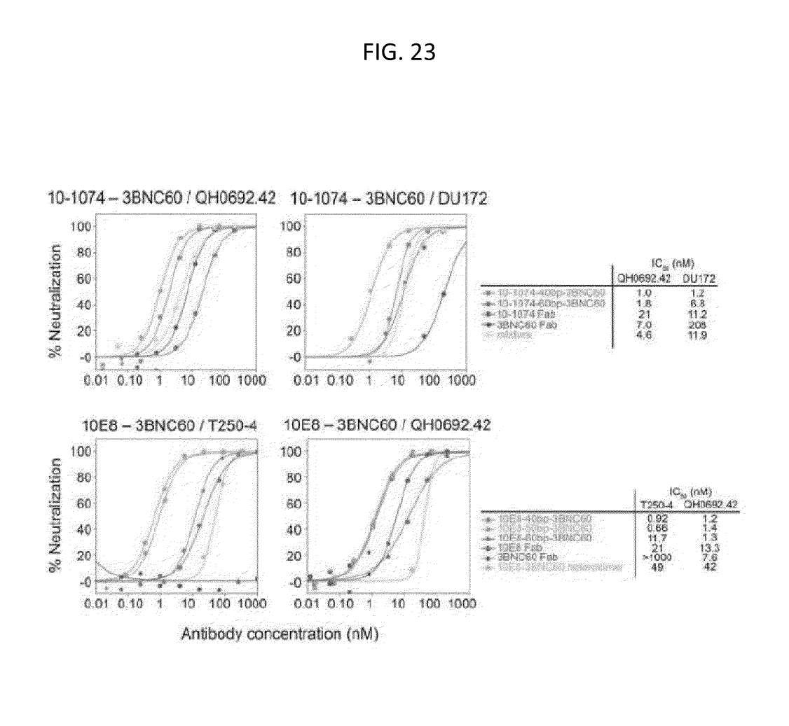

Neutralization data and IC₅₀ values of the neutralization data corresponding to varying lengths of dsDNA linkers for anti-HIV-1 homo-diFabs and hetero-diFabs are shown in FIGS. 9, 11A-11D and 15-23. From this analysis, effective ranges of dsDNA linker lengths for the homo-diFabs and hetero-diFabs were determined for increased viral neutralization.

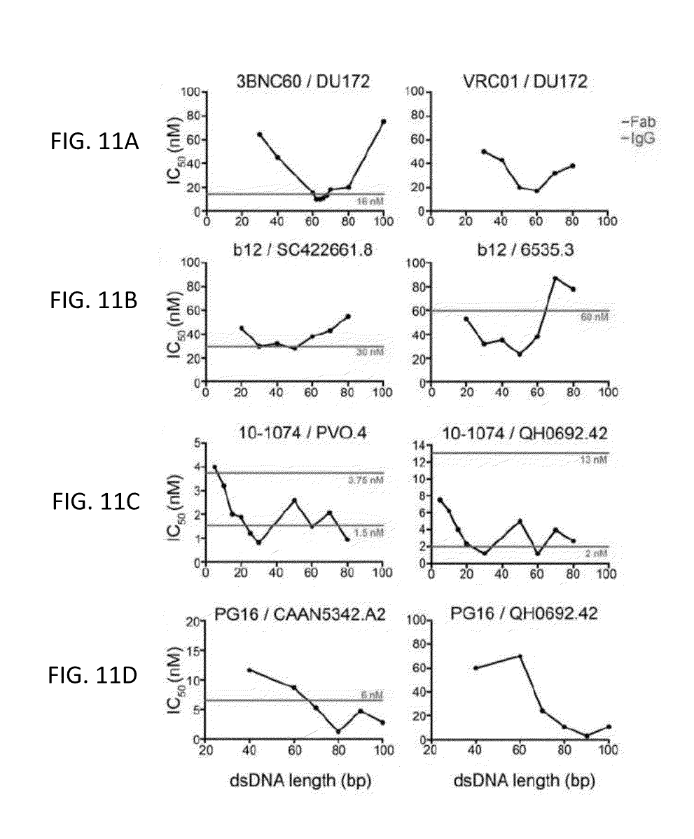

In some embodiments of the present invention, an anti-gp120 CD4 homo-diFab is conjugated with a linker molecule having a dsDNA length of about 40 to about 60 basepairs (bps) (FIG. 11A, 11B), corresponding to a length of about 130 Å to about 210 Å.

In some embodiments of the present invention, an anti-gp120 V3 homo-diFab is conjugated with a linker molecule having a dsDNA length of about 20 to about 36 bps (FIG. 11C), corresponding to a length of about 70 Å to about 120 Å.

In some embodiments of the present invention, an anti-gp120 V1V2 homo-diFab is conjugated with a linker molecule having a dsDNA length of about 65 to about 100 bps (FIG. 11D), corresponding to a length of about 221 Å to about 340 Å.

In some embodiments of the present invention, an anti-gp120 V1V2-CD4 hetero-diFab is conjugated with a linker molecule having a dsDNA length of about 24 to about 50 bps, corresponding to a length of about 80 Å to about 170 Å.

In some embodiments of the present invention, an anti-gp120 V3-CD4 hetero-diFab is conjugated with a linker molecule having a dsDNA length of about 18 to about 60 bps, corresponding to a length of about 60 Å to about 200 Å.

In some embodiments of the present invention, an anti-gp41-CD4 hetero-diFab is conjugated with a linker molecule having molecule having a dsDNA length of about 20 to about 62 bps, corresponding to a length of about 70 Å to about 210 Å.

The selection of dsDNA linker molecules of a particular length is not limited by the sequences disclosed in Table 1, as DNA nucleotides may be interchanged predictably as long as the sequence is analyzed for secondary structure features. The linker sequences disclosed in Table 1 may be modified with any basepair substitutions so long as the length and consensus region is maintained and sequences that result in secondary structures (e.g., stem loops, tetraloops, and pseudoknots) are not used. Sequences resulting in secondary structures are identified using any prediction tool software, such as, OligoAnalyzer, Integrated DNA Technologies (IDT).

Anti-HIV-1 diFabs Crosslinked with Protein Linkers

Using the desired linker lengths as determined with dsDNA, protein linker molecules of similar length and rigidity and flexibility may be designed to crosslink the anti-HIV-1 homo-diFabs and hetero-diFabs. Tetratricopeptide repeat (TPR) domains may be used to substitute for the dsDNA linker. TPR repeat domains are found in natural proteins and are effective protein linkers because the length of a set of tandem TPR domains corresponds predictably with the number of repeats. TPR domains in nature consist of three sets of a highly degenerate consensus sequence of 34 amino acids, often arranged in tandem repeats, formed by two alpha-helices forming an antiparallel amphipathic structure and a final C-terminal α-7 helix. The TPR repeat sequence tolerates minor amino acid variations at certain positions.

In some embodiments of the present invention, a protein linker molecule includes a TPR repeat, in which one TPR repeat is encoded by SEQ ID No: 41: AX₁AWYNLGNAYYKQGDYDEAIX₂YYQKALELDPX₃X₄ where X₁ is E, K, or S; X₂ is E or D; X₃ is R or N; and X₄ is S or N. In some embodiments of the present invention, a protein linker molecule includes from 3 to 30 TPR repeats. In some embodiments, a protein linker includes from 3 to 27 TPR repeats, from 3 to 24 TPR repeats, from 3 to 21 TPR repeats, from 3 to 18 TPR repeats, from 3 to 15 TPR repeats, from 3 to 12 TPR repeats, from 3 to 9 TPR repeats, or from 3 to 6 TPR repeats.

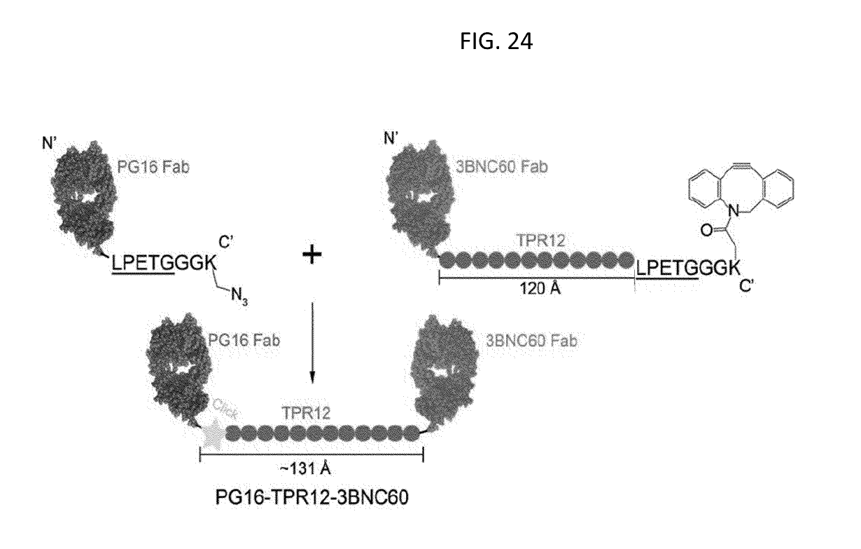

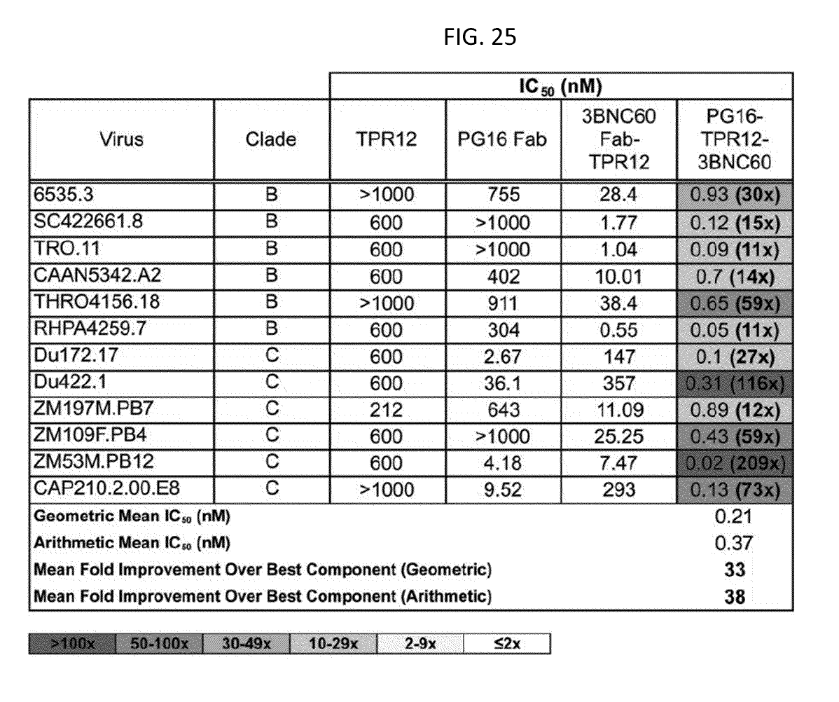

Selection of the number of TPR repeats correlates with the desired linker length for the corresponding homo-diFabs or hetero-diFabs. From the dsDNA linker analysis disclosed herein, effective linker molecules having improved neutralization have from 20 basepairs (bps) to 100 bps. As shown in FIG. 24, a linker molecule of 12 TPR repeats including linkers and conjugation moieties, approximates 131 angstroms (Å) which corresponds to about 40 bps of dsDNA. Accordingly, a 12 TPR linker molecule effectively crosslinks an anti-gp120 V1V2 (PG16) and anti-gp120 CD4(3BNC60) hetero-diFab as shown in FIG. 25. As disclosed herein, an anti-gp120 V1V2-CD4 hetero-diFab shows improved potency and neutralization when crosslinked with a linker molecule having a length of about 80 Å to about 170 Å. Accordingly, in some embodiments of the present invention, an anti-gp120 V1V2-CD4 hetero-diFab has a linker molecule including from 6 TPR domains up to 15 TPR domains.

As disclosed herein, an anti-gp120 CD4 homo-diFab shows improved potency and neutralization when crosslinked with a linker molecule having a length of about 130 Å to about 210 Å. Accordingly, in some embodiments of the present invention, an anti-gp120 CD4 homo-diFab has a linker molecule including from 12 TPR domains up to 20 TPR domains.

As disclosed herein, an anti-gp120 V3 homo-diFab shows improved potency and neutralization when crosslinked with a linker molecule having a length of about 70 Å to about 120 Å. Accordingly, in some embodiments of the present invention, an anti-gp120 V3 homo-diFab has a linker molecule including from 6 TPR domains up to 12 TPR domains.

As disclosed herein, an anti-gp120 V1V2 homo-diFab shows improved potency and neutralization when crosslinked with a linker molecule having a length of about 221 Å to about 340 Å. Accordingly, in some embodiments of the present invention, an anti-gp120 V1V2 homo-diFab has a linker molecule including from 18 TPR domains up to 30 TPR domains.

As disclosed herein, an anti-gp120 V3-CD4 hetero-diFab shows improved potency and neutralization when crosslinked with a linker molecule having a length of about 60 Å to about 200 Å. Accordingly, in some embodiments of the present invention, an anti-gp120 V3-CD4 hetero-diFab has a linker molecule including from 6 TPR domains to 18 TPR domains.

As disclosed herein, an anti-gp41-CD4 hetero-diFab shows improved potency and neutralization when crosslinked with a linker molecule having a length of about 70 Å to about 210 Å. Accordingly, in some embodiments of the present invention, an anti-gp41-CD4 hetero-diFab has a linker molecule including from 6 TPR domains up to 21 TPR domains.

In some embodiments of the present invention, small flexible linkers flank the TPR repeats. Examples of flexible linker segments include Gly-Gly-Gly-Gly-Ser (Gly4Ser)n motifs, where n is the number of repeats of the motif. As such, a protein linker molecule may include (Gly4Ser)₃-12TPR-(Gly4Ser)₃ in which three Gly4Ser motifs flank a set of 12 TPR repeats.

In some embodiments of the present invention, the pair of anti-HIV-1 Fabs are fused using sortase-catalyzed protein ligation and click chemistry as described in detail herein (e.g., Examples 4 and 6).

The following Examples are presented for illustrative purposes only, and do not limit the scope or content of the present application.

### EXAMPLES

Reference is made to Galimidi et al., 2015, Cell, 160:433-446 for disclosure of the methods and analysis presented in this disclosure, and reference is made to Klein et al., 2014, Prot. Eng. Design & Selection, 27:325-330 for disclosure of the TPR domain, the entire contents of both of which are incorporated herein by reference.

### Example 1

### Homo-diFabs Exhibit Length-Dependent Avidity Effects Consistent with Intra-Spike Crosslinking

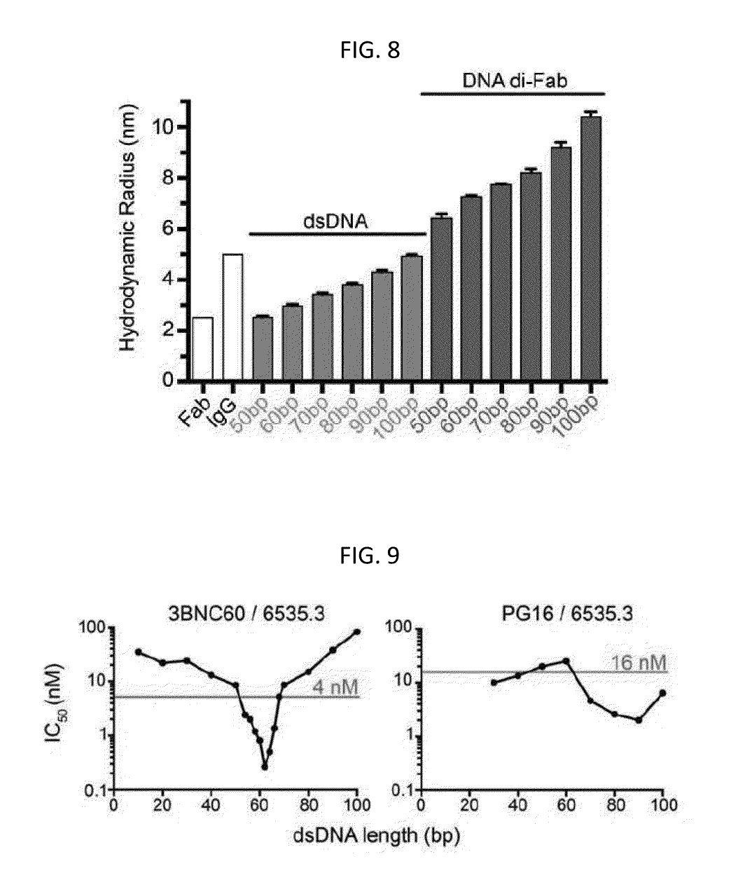

Fabs were modified to contain a free thiol and then conjugated to maleimide-activated single-stranded DNA (ssDNA) (FIG. 4). Different lengths of dsDNA (designed to lack secondary structures were annealed with and ligated to the ssDNA-Fab conjugates to create homo- or hetero-diFabs, in which the two Fabs were the same or different, respectively. (Zadeh et al., 2011, J. Comput. Chem. 32:170-173, the entire contents of which are incorporated herein by reference.) Dynamic light scattering confirmed that conjugates with longer DNA bridges were more extended (FIG. 8), supporting the use of double stranded DNA (dsDNA) as a ruler. Inter-Fab distances calculated from dsDNA lengths were regarded as approximate because the DNA linkers included short regions of ssDNA (persistence length 22 Å) to permit orientational flexibility. (Chi, et al., 2013, Physica A: Statistical Mechanics and its Applications 392, 1072-1079, the entire contents of which are incorporated herein by reference.)

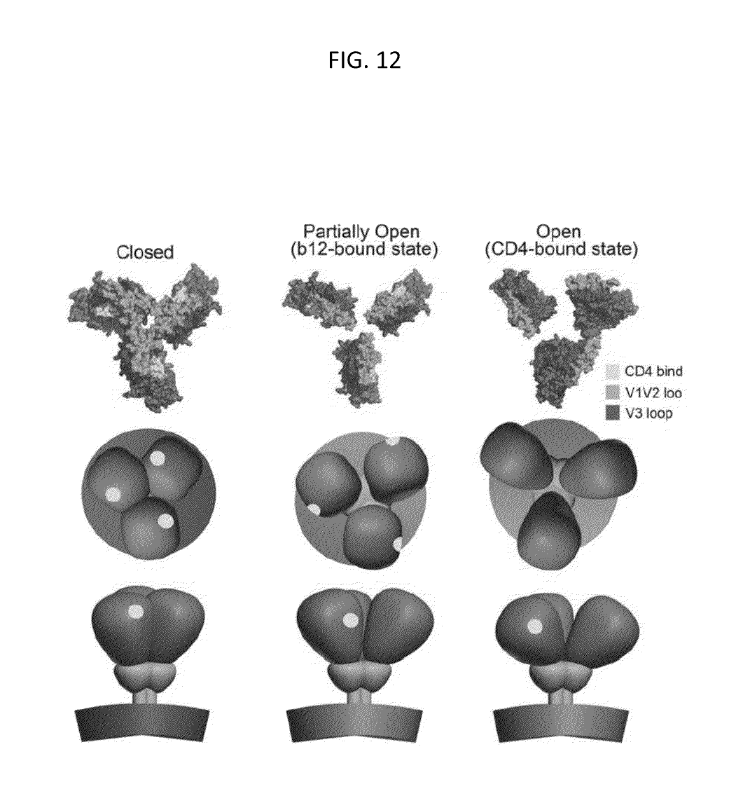

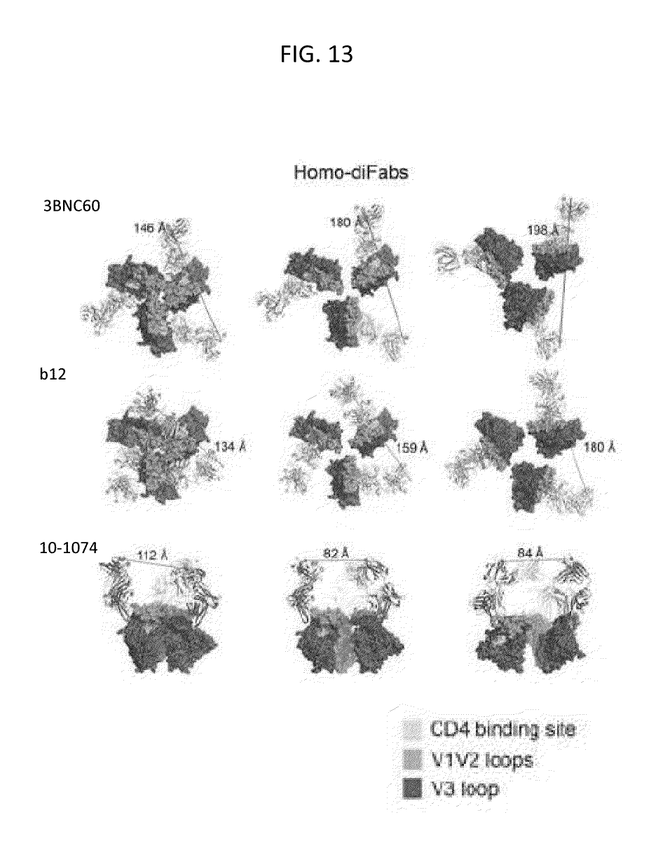

The optimal range of dsDNA linkers for a homo-diFab constructed from 3BNC60 (a broad neutralizing antibody (Nab) against the CD4 binding site (CD4bs) on the gp120 subunit of Env was determined by evaluating homo-diFabs with different dsDNA lengths using in vitro neutralization assays. The 50% inhibitory concentrations (IC₅₀s) against HIV-1 strain 6535.3 depended on the dsDNA length, with the most potent homo-diFab containing a bridge of 62 bp (211 Å) (FIGS. 8, 9, 10, 11A-11D). (Scheid et al. 2011, Science 334, 1289-1293, the entire contents of which are incorporated herein by reference.) This length is close to the predicted distance (approximately 198 Å) between the C-termini of adjacent 3BNC60 Fabs bound to the open structure of an HIV-1 trimer. (Merk et al., 2013, Curr Opin Struct Biol 23, 268-276, the entire contents of which are herein incorporated by reference.) (FIGS. 12, 13, 14). Bridge lengths of approximately 60 bp also exhibited the best potencies for 3BNC60 homo-diFabs against DU172.17 HIV-1 and for homo-diFabs constructed from VRC01, a related CD4bs bNAb (FIGS. 11A-11D). (Wu et al., 2010, Science, 329:856-861, the entire contents of which are herein incorporated by reference.) The approximate 100-fold increased potency of 3BNC60-62 bp-3BNC60 compared with 3BNC60 IgG against HIV-1 6535.3 (FIG. 9) suggested synergy resulting from avidity effects due to bivalent binding. The bivalent interaction likely resulted from intra-spike crosslinking rather than inter-spike crosslinking since the latter should not manifest with a sharp length-dependence because inter-spike distances are variable within and between virions. (Liu et al., 2008, Nature 455, 109-113; and Zhu et al., 2006, Nature 441, 847-852, the entire contents of both of which are incorporated herein by reference.)

To formally assess the extent to which inter-spike crosslinking could contribute to synergy, homo-diFabs constructed from the V1V2 loop-specific bNAb PG16 (which cannot crosslink within a single spike because only one anti-V1V2 Fab binds per Env trimer were evaluated. (Walker et al., 2009, Science, 326:285-289; Julien, et al., 2013, Proc Natl Acad Sci USA 110, 4351-4356, the entire contents of all of which are incorporated herein by reference.) PG16 homo-diFabs with different dsDNA bridges did not exhibit length-dependent neutralization profiles against strain 6535.3 (FIG. 9) and other viral strains (FIG. 11D). However, increased potencies were observed for PG16 homo-diFabs with greater than or equal to (≧) 70 bp or 80 bp (greater than or equal to (≧) 248 Å or 272 Å) bridges, perhaps reflecting increased inter-spike crosslinking with longer separation distances (FIG. 9; FIG. 11D).

### Example 2

### Comparison of homo-diFabs and Intra-Spike Crosslinking

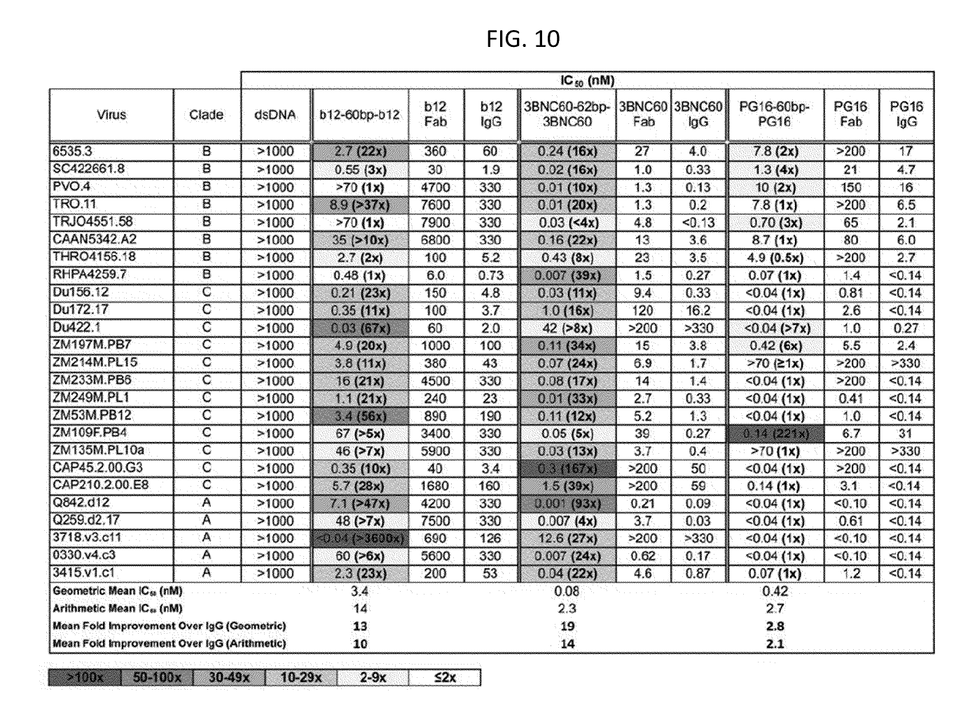

To evaluate the potential for intra-spike crosslinking across different viral strains, homo-diFabs designed to be capable (b12 and 3BNC60) or incapable (PG16) of intra-spike crosslinking (FIG. 10) were compared. To minimize inter-spike crosslinking, the homo-diFabs were constructed with 60-62 bp bridges. The b12-60 bp-b12 homo-diFab exhibited increased potency compared with b12 IgG in 21 of 25 strains in a cross-clade panel of primary HIV-1, with potency increases greater than or equal to (≧)10-fold for 16 strains and a geometric mean potency increase of 22-fold. 3BNC60-62 bp-3BNC60 showed even more consistent synergy, being more potent than 3BNC60 IgG against all 25 strains tested, with greater than or equal to (≧)10-fold increases for 20 strains and a mean increase of 19-fold. By contrast, the PG16-60 bp-PG16 homo-diFab showed potency increases compared with PG16 IgG against only six strains, with relatively small (2- to 7-fold) increases in five strains and an overall 2.8-fold mean potency change.

### Example 3

### Hetero-diFabs Exhibit Dramatic Potency Increases Consistent with Intra-Spike Crosslinking

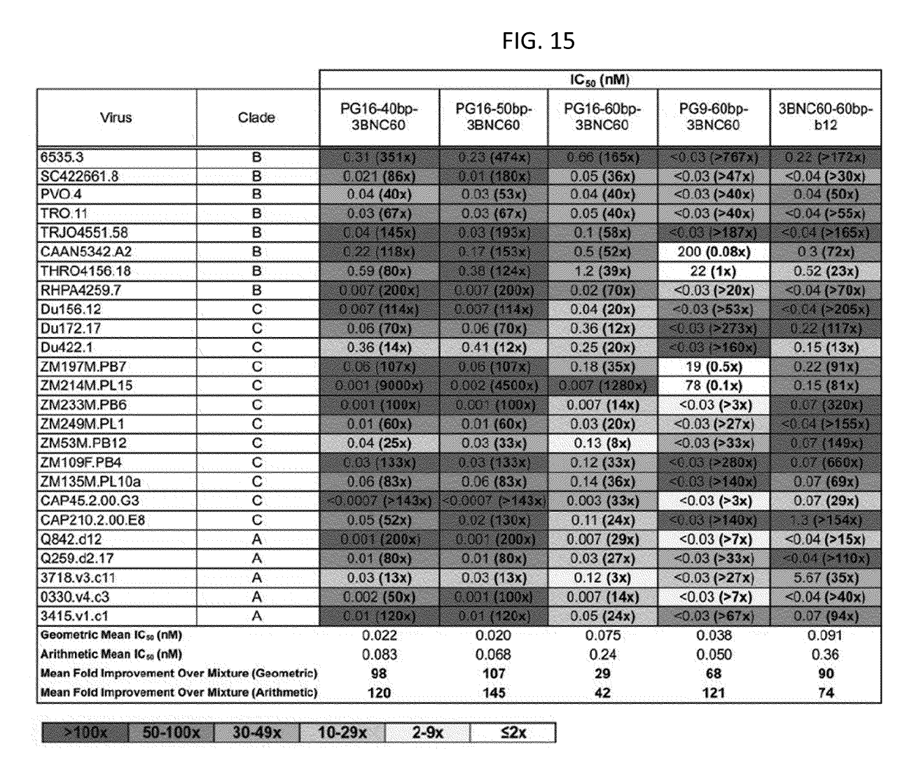

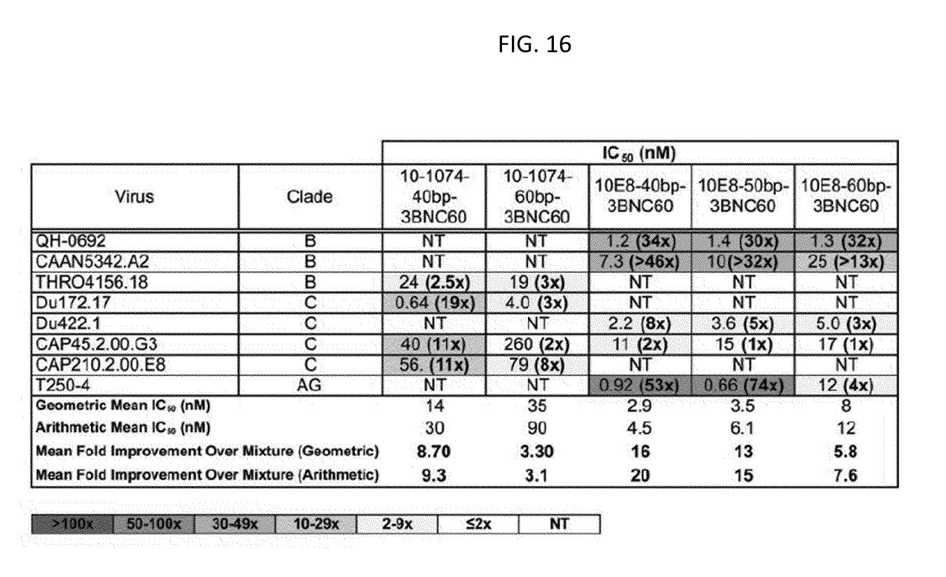

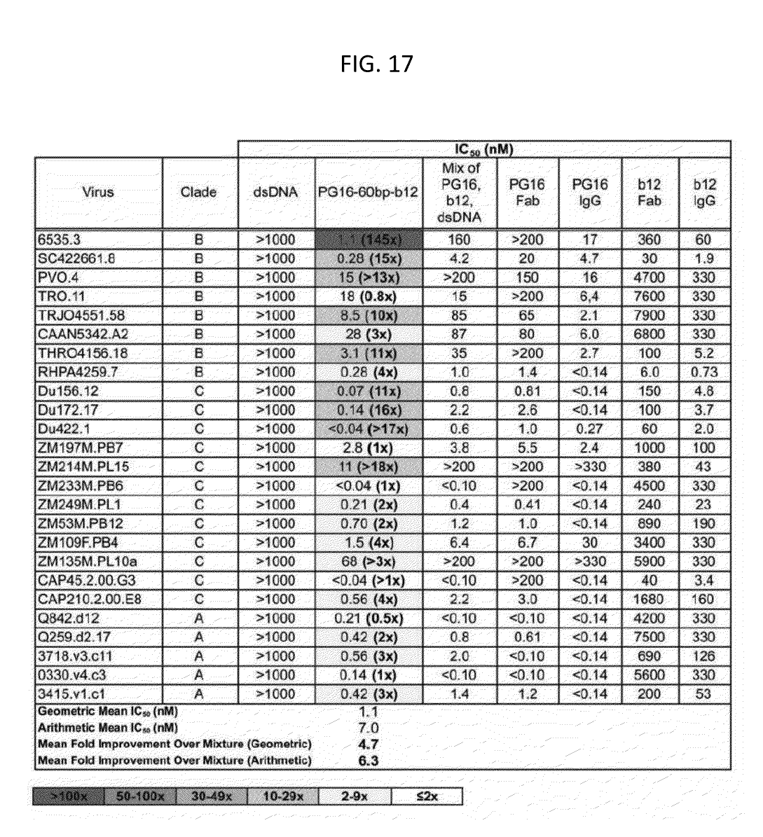

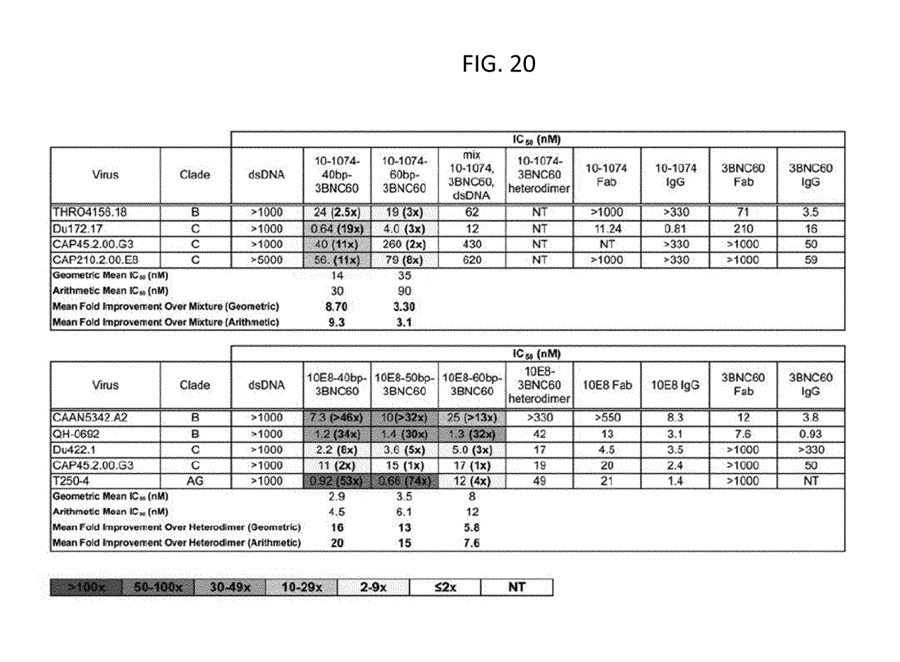

To determine whether heterotypic bivalent binding can produce synergy and to measure distances between epitopes, dsDNA was used to link Fabs recognizing different epitopes on gp120. Hetero-diFabs were constructed with Fabs from V1V2 (PG16 or PG9) and CD4bs (b12 or 3BNC60) bNAbs linked with 60 bp dsDNA bridges. PG16-60 bp-b12 hetero-diFabs were evaluated in neutralization assays against HIV-1 strains SC4226618 (more sensitive to b12 than PG16) and CAP210 (more sensitive to PG16 than b12). (Walker et al., 2009 supra, Roben et al., 1994, J. Virol. 68: 4821-4828; Scheid et al., 2011, Science, 333:1633-1637, the entire contents of all of which are herein incorporated by reference.) According to the model being tested, in the absence of synergistic binding; i.e., when only one Fab can bind to a spike at a time, a hetero-diFab would be no more potent than a non-covalent mixture of the dsDNA and the two Fabs against each viral strain, whereas synergistic binding would result in avidity effects exhibited by increased potency of the hetero-diFab. For both viral strains, the PG16-60 bp-b12 hetero-diFab was approximately 10-fold more potent than the mixture of Fabs plus dsDNA or the more potent of the two Fabs alone (FIGS. 15, 16, 17, 18, 19, 20, 21, 22). To more systematically explore potential synergy, PG16-60 bp-b12 was evaluated against a 25-member panel of HIV-1 strains, finding synergistic effects (between 2- and 145-fold more potent than the corresponding non-covalent mixture for most strains; geometric mean improvement of 4.7-fold) (FIG. 17). When Fabs from PG16 or PG9 were combined with a more potent CD4bs-recognizing bNAb (3BNC60), the resulting hetero-diFabs exhibited greater synergy—several examples of greater than (>) 150-fold improvement for PG16-60 bp-3BNC60 and PG9-60 bp-3BNC60 and geometric mean potency improvements of 29- and 68-fold, respectively (FIGS. 15, 16, 18, 19). Other hetero-diFabs, constructed with combinations of Fabs recognizing the CD4bs (3BNC60 (Scheid et al., 2011, supra)), the gp120 V3 loop (10-1074 (Mouquet et al., 2012)), and a gp41 epitope (10E8), also showed synergistic effects (FIGS. 15 and 20), and a 3BNC60-60 bp-b12 hetero-diFab exhibited up to 660-fold synergy and a geometric mean potency increase of 90-fold (FIGS. P, V) (Huang et al., 2012, Nature, 491:406-412, the entire contents of which are herein incorporated by reference. In contrast, analogous IgG heterodimers constructed with two different Fabs linked to a single Fc did not show synergy when evaluated against the same viruses, demonstrating that synergistic effects required optimal separation distances that permitted each Fab to achieve its specific binding orientation (FIGS. 16, 17, 18, 19, 20, 21, and 22). These data show that hetero-diFabs can achieve synergy through simultaneous recognition of two different epitopes on the same HIV-1 Env trimer.

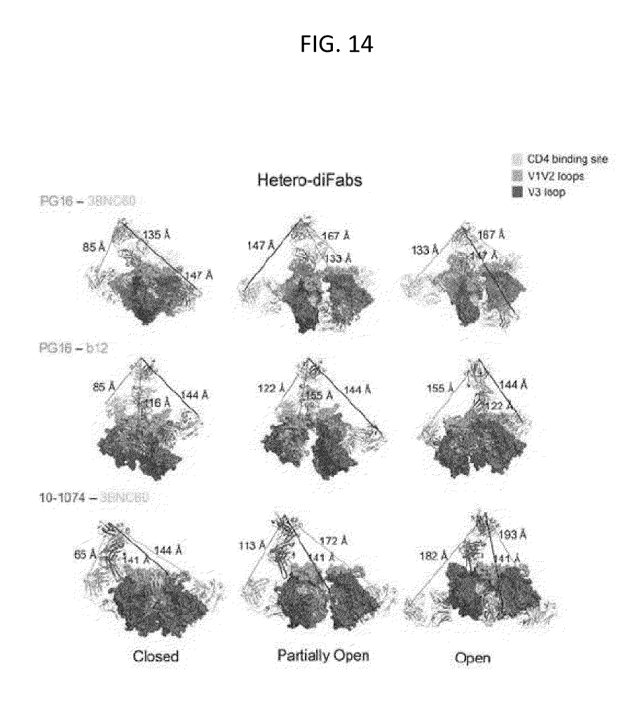

To more precisely define optimal intra-epitope separation distances, hetero-diFabs were evaluated with different bridge lengths, finding length-dependent synergy effects. For example, PG16-3BNC60 hetero-diFabs with 40 bp and 50 bp dsDNA bridges showed improved neutralization potencies when compared to the 60 bp (204 Å) version, achieving greater than or equal to (≧) 100-fold potency increases against over half of the tested strains and geometric mean improvements of 98- and 107-fold respectively (FIGS. 15, 16, 20). The 40 bp and 50 bp bridges (136 Å and 170 Å, respectively) corresponded to the approximate separation distances between PG16 and 3BNC60 Fabs when bound to the same gp120 within a trimer (147 Å) or to neighboring protomers within open or partially-open trimers (167 Å) (FIG. 14). In a second length dependency example, 10-1074-40 bp-3BNC60 was more potent than 10-1074-60 bp-3BNC60 (FIGS. 15, 16, 20). The approximate 136 Å distance between the two Fabs in 10-1074-40 bp-3BNC60 corresponded to the approximate separation between these Fabs bound to the same gp120 (141 Å), while 60 bp more closely approximated Fabs bound to neighboring protomers on an open trimer (193 Å) (FIG. 14). The 40 bp and 50 bp versions of 10E8-3BNC60 showed consistent synergy (FIGS. 15, 16, 20); however, the lack of structural information concerning 10E8 binding to Env trimer hindered interpretation of 10E8-containing hetero-diFabs.

### Example 4

### A hetero-diFab Constructed with a Protein Linker Exhibits Synergistic Potency Increases

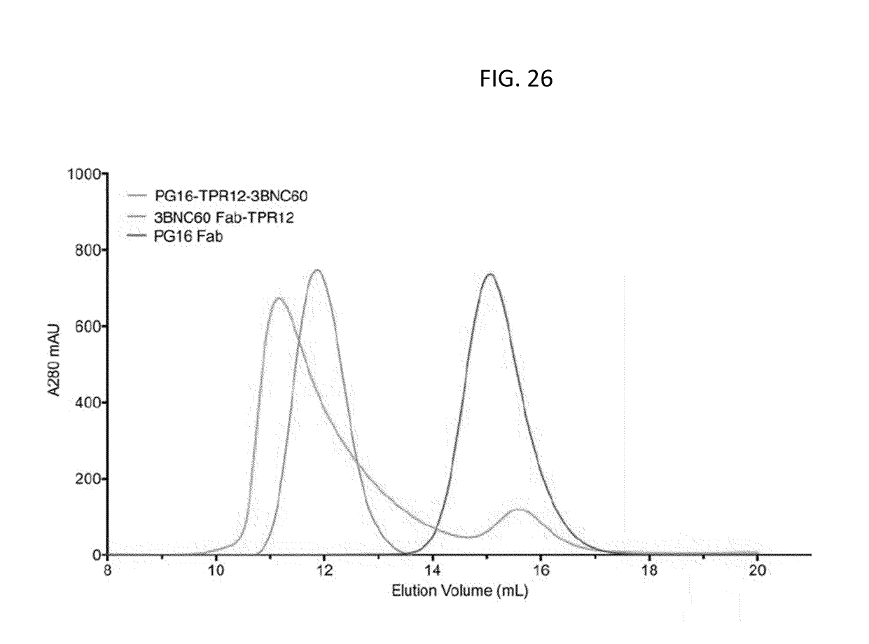

Bivalent molecules involving dsDNA linkers were effective for demonstrating synergistic neutralization, but a protein reagent would be preferable as an anti-HIV-1 therapeutic. A series of protein linkers of various lengths and rigidities that can mimic the properties of different lengths of dsDNA are described in Klein et al., 2014, the entire contents of which is herein incorporated by reference. As such, it is possible to substitute a comparable protein linker for an optimal dsDNA bridge to create a protein reagent capable of simultaneous binding to two different epitopes on a single HIV-1 spike trimer. As an example, sortase-catalyzed protein ligation and click chemistry was used to construct a bivalent reagent analogous to PG16-40 bp-3BNC60 by substituting the dsDNA linker with 12 domains of a designed tetratricopeptide-repeat (TPR) protein (Witte et al., 2013, Nat. Protoc. 8:1808-1819; and Kajander et al., 2007, Acta Crystallographica Section D-Biological Crystallography 63, 800-811, the entire contents of both of which are herein incorporated by reference.) (FIGS. 24, 25, 26). A TPR linker was chosen because tandem repeats of TPR domains form a rigid rod-like structure whose length corresponds predictably with the number of repeats, with each domain contributing approximately 10 Å (Kajander et al., 2007, supra). PG16 Fab was expressed with a C-terminal sortase signal, and the C-terminus of the 3BNC60 Fab was modified to include twelve TPR repeats and a sortase signal. The tagged Fabs were covalently attached to peptides containing click handles using sortase-catalyzed ligation, and then incubated to allow the click reaction to form PG16 Fab linked to 3BNC60 Fab by twelve TPR repeats (PG16-TPR12-3BNC60). Together with the remnants of the click handles, the linker would occupy approximately 131 Å, which is approximately the same length as the dsDNA linker in PG16-40 bp-3BNC60 reagent (FIGS. 24, 25, 26). The protein-based molecule, PG16-TPR12-3BNC60, exhibited between 11- and >200-fold synergy against 12 primary HIV-1 strains (FIG. 25; 33-fold geometric mean increased potency).

### Example 5

### Simulations of the Effects of Avidity on IgG Binding to Tethered Antigens

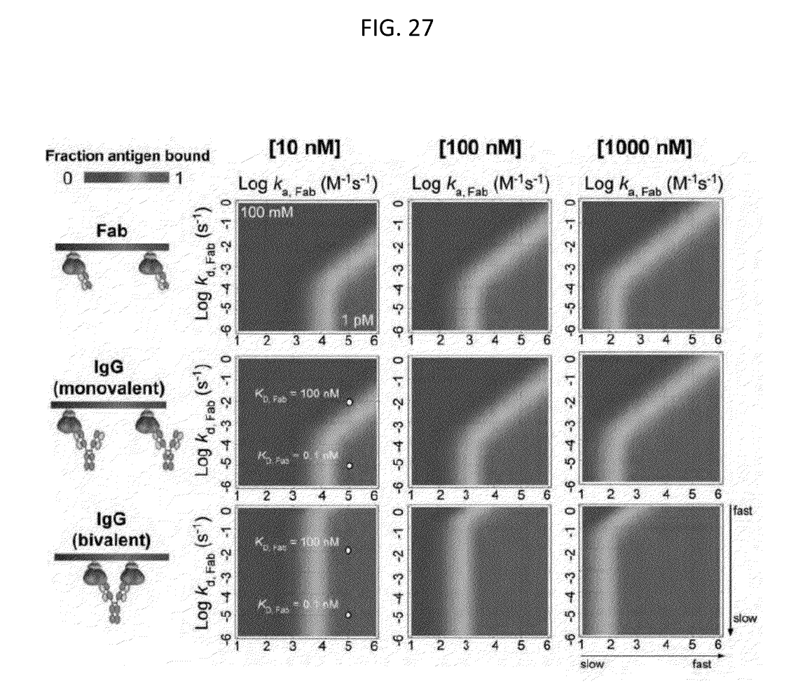

In order to better understand the effects of avidity arising from bivalent binding of IgGs to antigens tethered to a surface such as a viral membrane, modeling software was used to simulate the saturation of surface-bound antigens by monovalent Fabs and bivalent IgGs. A 1-hour incubation time was chosen based upon conditions under which in vitro neutralization assays are conducted (Montefiori, 2005, Current Protocols in Immunology, edited by John E. Coligan et al., Chapter 12, Unit 12 11, the entire contents of which are herein incorporated by reference.) The density of the tethered antigens and the concentrations of Fab or IgG were varied in order to investigate a range of intrinsic association and dissociation rate constants for the binding interaction. The fraction of antigen bound by a Fab or IgG was calculated as a function of on- and off-rates (kₐ and k_{d}), whose ratio (k_{d}/kₐ) is equal to the affinity (K_{D}, or equilibrium dissociation constant). Saturation by Fabs (top row) was compared, as well as IgGs in which only monovalent binding was permitted (center row), and IgGs that bound bivalently through crosslinking of neighboring antigens (bottom row) (FIG. 27). As expected, saturation by Fabs and IgGs was nearly identical for monovalent binding conditions (FIG. 27, first two rows). By contrast, across a range of input concentrations, there were kₐ and k_{d} combinations for IgGs binding bivalently that exhibited saturation binding under conditions in which monovalent Fabs and IgGs binding monovalently did not (FIG. 27, bottom row). Thus, consistent with experimental results in the palivizumab/RSV system, the simulations suggested that bivalency through crosslinking can rescue binding of IgGs whose Fabs exhibit weak binding affinities as a result of fast dissociation rate constants, whereas IgGs whose Fabs exhibit high affinities because of slow dissociation rates did not display strong avidity enhancement. (Wu et al., 2005, J. Mol. Biol., 350: 126-144, the entire contents of which are herein incorporated by reference.)

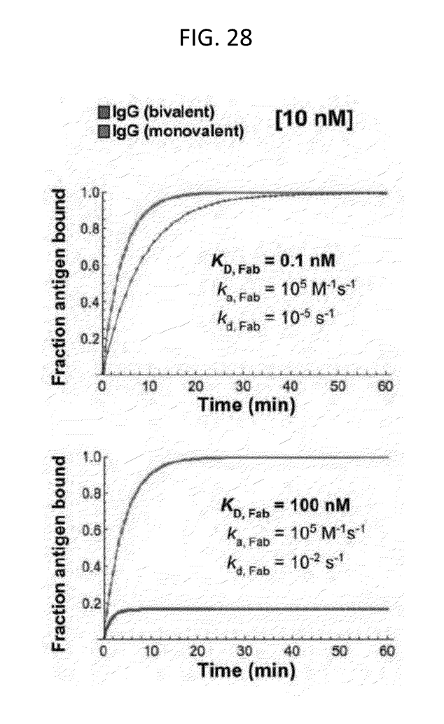

The simulations also demonstrate that the effects of avidity on binding are a complicated mixture of kinetics, input concentration, and incubation time. At any particular concentration, the threshold at which avidity is observed is controlled by kinetics rather than affinity because different combinations of kinetic constants yield the same K_{D}. The kinetic threshold at which avidity effects are observed varies depending on the difference between the input concentration and the K_{D}. For concentrations near or below the K_{D}, there is a kinetic threshold such that for on- and off-rates slower than ˜10³ M⁻¹s⁻¹ and ˜10⁻⁵ s⁻¹, respectively, avidity enhancement is not observed (FIGS. 27 and 28). The binding reactions are also affected by the length of incubation, such that the lower the input concentration, the longer it takes to reach saturation (FIGS. 27 and 28).

It is noted that the simulations only model binding interactions, whereas the homo- and hetero-diFabs were evaluated for their ability to enhance neutralization of viral infectivity, which is a process more complicated than binding. For example, neutralization mechanisms may involve conformational changes in Env that were not accounted for in the binding simulation. In addition, kinetics constants for antibody-mediated neutralization of HIV-1 are not known; nor is the fraction of Env spikes on a virion that are required for neutralization or for fusion. In any case, it appears that the kinetic properties of the bNAb Fab components in the disclosed reagents were appropriate to realize avidity-enhanced neutralization since hetero-diFab reagents displayed approximately 100-fold mean improved neutralization potencies. The data disclosed herein therefore support the hypothesis that intra-spike crosslinking by anti-HIV-1 binding molecules represents a valid strategy for increasing potency and resistance to HIV-1 Env mutations.

### Example 6

### Experimental Procedures

Expression and Purification of Fabs.

Genes encoding IgG light chain genes were modified by site-directed mutagenesis to replace Cys263_{Light Chain}, the C-terminal cysteine that forms a disulfide bond with Cys233_{Heavy Chain}, with a serine. Modified light chain genes and genes encoding 6×-His- or StrepII-tagged Fab heavy chains (V_{H}-C_{H}1-tag) were subcloned separately into the pTT5 mammalian expression vector (NRC Biotechnology Research Institute). Fabs were expressed by transient transfection in HEK 293-6E (NRC Biotechnology Research Institute) cells and purified from supernatants by Ni-NTA or StrepII affinity chromatography followed by size exclusion chromatography in PBS pH 7.4 using a Superdex 200 10/300 or Superdex 200 16/600 column (Amersham Biosciences), as described in Diskin et al., 2011, 334:1289-1293, the entire contents of which are herein incorporated by reference.

IgG Heterodimer Expression and Purification.

Bispecific IgGs were constructed using “knobs-into-holes” mutations (Thr366Trp on one heavy chain, and Thr366Ser, Leu368Ala, and Tyr407Val on the other heavy chain) to promote Fc heterodimerization, and crossover of the heavy and light chain domains of one half of the bispecific IgG to prevent light chain mispairing. Heterodimerizing leucine zipper sequences followed by either a 6×-His or Strep II tag sequence were added to the C-termini of the heavy chains. The V_{H} domain on one heavy chain of each heterodimer was replaced by the V_{L} domain, and the corresponding light chain was constructed with the V_{H} domain joined to the C_{L} domain. Heterodimeric IgGs were expressed by transient transfection and isolated from supernatants by Protein A chromatography followed by Strep II and Ni-NTA chromatography. Heterodimers were further purified by size exclusion chromatography using a Superdex 200 10/300 or 16/600 column (Amersham Biosciences) equilibrated in PBS pH 7.4.

In Vitro Neutralization Assays.

Neutralization of pseudoviruses derived from primary HIV-1 isolates was monitored by the reduction of HIV-1 Tat-induced luciferase reporter gene expression in the presence of a single round of pseudovirus infection in TZM-bl cells as described (Montefiori, 2005, supra). In some cases, DEAE-dextran, an additive used to enhance viral infection of target cells (Montefiori, 2005, supra), led to false positive neutralization signals for dsDNA alone and for dsDNA-containing reagents, presumably because of interactions between dextran and DNA. (Maes et al., 1967, Nucleic Acids and Protein Synthesis, 134:269-276, the entire contents of which are herein incorporated by reference.) Dextran was eliminated from assays in which the dsDNA linker alone reduced infectivity, in which case the pseudovirus concentration was increased by 2.5-40-fold, allowing for comparable infectivity as in the presence of dextran.

Pseudoviruses were generated by co-transfecting HEK293T cells with vectors encoding Env and a replication-deficient HIV-1 backbone as described (Montefiori, 2005) or obtained from the Fraunhofer Institut IBMT (6535.3, CAAN5342, CAP45, CAP210.200.E8, DU172, DU422, QH-0692, THR04156.18, TRO.11, ZM53, ZM214, ZM233, ZM249). Some of the neutralization data were derived from neutralization assays that were prepared by a Freedom EVO® (Tecan) liquid handler. Reagents (prepared as 3-, 4-, or 5-fold dilution series; each concentration in duplicate or triplicate) were incubated with 250 (when DEAE-dextran was added) or >1000 viral infectious units at 37° C. for one hour prior to incubation with reporter cells (10,000/well) for 48 hours. Luciferase levels were measured from a cell lysate using an Infinite 200 Pro microplate reader (Tecan) after addition of BrightGlo (Promega). Data were fit by Prism (GraphPad) using nonlinear regression to derive IC₅₀ values. IC₅₀s derived from independent replicates of manual and robotic assays generally agreed within 2-4 fold. Average IC₅₀ values reported in the figures and tables are geometric means calculated using the formula (Πaᵢ)^{(1/n)}; i=1, 2, . . . , n. Geometric means are suitable statistics for data sets covering multiple orders of magnitude, as is the case for neutralization data across multiple viral strains. Fold improvements were calculated as the ratio of the geometric mean IC₅₀ values for the reagents being compared.

DNA Conjugation to Fabs.

DNA was conjugated to free thiol-containing Fabs using a modified version of a previously-described protocol as described in Hendrickson et al., 1995, Nucleic Acids Research, 23: 522-529, the entire contents of which are herein incorporated by reference. Briefly, Fabs were reduced in a buffer containing 10 mM TCEP-HCl pH 7-8 for two hours, and then buffer exchanged three times over Zeba desalting columns (Thermo Scientific). The percentage of reduced Fab was determined using Invitrogen's Measure-IT Thiol Assay. Concurrently, a 5-20 base ssDNA containing a 5′ amino group (Integrated DNA Technologies, IDT-DNA) was incubated with a 100-fold molar excess of an amine-to-sulfhydryl crosslinker (Sulfo-SMCC; Thermo Scientific) for 30 minutes to form a maleimide-activated DNA strand, which was buffer exchanged as described above. The reduced Fab and activated ssDNA were incubated overnight, and the Fab-ssDNA conjugate was purified by Ni-NTA or StrepII affinity chromatography (GE Biosciences) to remove unreacted Fab and ssDNA.

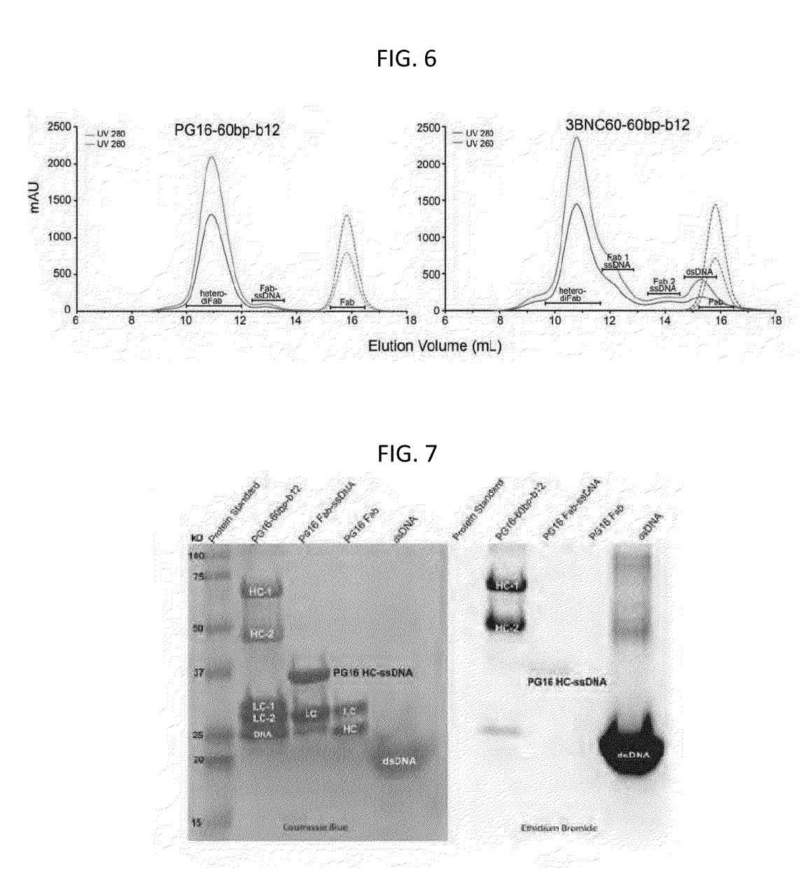

ssDNA was synthesized, phosphorylated, and PAGE purified by Integrated DNA Technologies. For di-Fabs containing dsDNA bridges longer than 40 bp, complementary ssDNAs were annealed by heating (95° C.) and cooling (room temperature) to create dsDNA containing overhangs complementary to the Fab-ssDNA conjugates. dsDNA was purified by size exclusion chromatography (Superdex 200 10/300) and incubated overnight with the corresponding tagged Fab-ssDNA conjugates. Homo- and hetero-diFab reagents were purified by Ni-NTA and StrepII affinity chromatography when appropriate to remove free DNA and excess Fab-ssDNA conjugates, treated with T4 DNA ligase (New England Biolabs), and purified again by size exclusion chromatography (FIG. 6). To make di-Fabs containing dsDNA bridge lengths less than 40 bp, two complementary ssDNA-conjugated Fabs were incubated at 37° C. without a dsDNA bridge and then purified as described above. Protein-DNA reagents were stable at 4° C. for greater than 6 months as assessed by SDS-PAGE.

Bridge and linker sequences are listed in Table 1.

TABLE 1

| | Linker | SEQ | |
|---|---|---|---|
| | Lenght | ID | |
| DNA type | (bp) | NO. | DNA sequence 5′ to 3′ |
| Fab 1 | 32 | 1 | 5-/5AmMC6/TTT TTT TTT TTT CTT TGT TCT TAT TCT CTG CT-3 |
| ssoligo | | | |
| Fab 2 | 32 | 2 | 5-/5AmMC6/AAG AGA GAG AAA AGG AAG AAG GGA AGA AGA GG-3 |
| ssoligo | | | |
| linker | | | |
| 10 bp bridge | 10 | 3 | 5-/5AmMC6/TTT TTT TTT TTT GGA CGA AGT C-3 |
| and linker | | 4 | 5-/5AmMC6/AAG AGA GAG AAA GAC TTC GTC C-3 |
| 15 bp bridge | 15 | 5 | 5-/5AmMC6/TTT TTT TTT TTT GGA CGA AGT CCA ACC-3 |
| and linker | | 6 | 5-/5AmMC6/AAG AGA GAG AAA GGT TGG ACT TCG TCC-3 |
| 20 bp bridge | 20 | 7 | 5-/5AmMC6/TTT TTT TTT TTT CGT GGT CAT GAG CCG GGA CG-3 |
| and linker | | 8 | 5-/5AmMC6/AAG AGA GAG AAA CGT CCC GGC TCA TGA CCA CG-3 |
| 25 bp bridge | 25 | 9 | 5-/5AmMC6/TTT TTT TTT TTT CGT GGT CAT GAG CCG GGA CGA |
| and linker | | | AGT C-3 |
| | | 10 | 5-/5AmMC6/AAG AGA GAG AAA GAC TTC GTC CCG GCT CAT GAC |
| | | | CAC G-3 |
| 30 bp bridge | 30 | 11 | 5-/5AmMC6/TTT TTT TTT TTT CGT GGT CAT GAG CCG GGA CGA |
| and linker | | | AGT CCA ACC-3 |
| | | 12 | 5-/5AmMC6/AAG AGA GAG AAA GGT TGG ACT TCG TCC CGG CTC |
| | | | ATG ACC ACG-3 |
| 40 bp bridge | 40 | 13 | 5-/5Phos/GAG GAC TAT CCG GCG CCG TCC CTC TTC TTC CCT |
| and linker | | | TCT TCC T-3 |
| | | 14 | 5-/5Phos/GAC GGC GCC GGA TAG TCC TCA GCA GAG AAT AAG |
| | | | AAC AAA G-3 |
| 50 bp bridge | 50 | 15 | 5-/5Phos/TGG GCG ACT CGA CGG CGC CGG ATA GTC CTC AGC |
| and linker | | | AGA GAA TAA GAA CAA AG-3 |
| | | 16 | 5-/5Phos/GAG GAC TAT CCG GCG CCG TCG AGT CGC CCA CCT |
| | | | CTT CTT CCC TTC TTC CT-3 |
| 60 bp bridge | 60 | 17 | 5-/5Phos/T TCT TTC TTT CCT CCT TCT CCC TCT TCT TCC CTT |
| and linker | | | CTT CCT-3 |
| | | 18 | 5-/5Phos/G AGA AGG AGG AAA GAA AGA AAG CAG AGA ATA AGA |
| | | | ACA AAG-3 |
| 70 bp bridge | 70 | 19 | 5-/5Phos/TTT TTT TTT TTT CGT GGT CAT GAG CCG GGA CG-3 |
| and linker | | 20 | 5-/5Phos/AGC CTT ACT GGT GGT GCC ACT GGG CGA CTC GAC |
| | | | GGC GCC GGA TAG TCC TCA GCA GAG AAT AAG AAC AAA G-3 |
| 80 bp bridge | 80 | 21 | 5-/5Phos/GAG GAC TAT CCG GCG CCG TCG AGT CGC CCA GTG |
| and linker | | | GCA CCA CCA GTA AGG CTT ATC GCA TGT CCT CTT CTT CCC |
| | | | TTC TTC CT-3 |
| | | 22 | 5-/5Phos/ACA TGC GAT AAG CCT TAC TGG TGG TGC CAC TGG |
| | | | GCG ACT CGA CGG CGC CGG ATA GTC CTC AGC AGA GAA TAA |
| | | | GAA CAA AG-3 |
| 90 bp bridge | 90 | 23 | 5-/5Phos/GAG GAC TAT CCG GCG CCG TCG AGT CGC CCA GTG |
| and linker | | | GCA CCA CCA GTA AGG CTT ATC GCA TGT AAG TTG CAC CCC |
| | | | TCT TCT TCC CTT CTT CCT-3 |
| | | 24 | 5-/5Phos/GGT GCA ACT TAC ATG CGA TAA GCC TTA CTG GTG |
| | | | GTG CCA CTG GGC GAC TCG ACG GCG CCG GAT AGT CCT CAG |
| | | | CAG AGA ATA AGA ACA AAG-3 |
| 100 bp bridge | 100 | 25 | 5-/5Phos/GAG GAC TAT CCG GCC CCG TCG AGT CGC CCA GTG |
| and linker | | | GCA CCA CCA GTA AGG CTT ATC GCA TGT AAG TTG CAC CCC |
| | | | CAT CCT CCC CTC TTC TTC CCT TCT TCC T-3 |
| | | 26 | 5-/5Phos/GGA GGA TGG GGG TGC AAC TTA CAT GCG ATA AGC |
| | | | CTT ACT GGT GGT GCC ACT GGG CGA CTC GAC GGG GCC GGA |
| | | | TAG TCC TCA GCA GAG AAT AAG AAC AAA G-3 |

Characterization of DNA-Fab Reagents.

Fractions from the center of an SEC elution peak were concentrated using Amicon Ultra-15 Centrifugal Filter Units (Millipore) (MW cutoff=10 kDa) to a volume of 500 μL, and DLS measurements were performed on a DynaPro® NanoStar™ (Wyatt Technology) using the manufacturer's suggested settings. Hydrodynamic radii were determined as described (Dev and Surolia, 2006). Briefly, a nonlinear least squares fitting algorithm was used to fit the measured correlation function to obtain a decay rate. The decay rate was converted to the diffusion constant that can be interpreted as the hydrodynamic radius via the Stokes-Einstein equation.

Hetero-diFab with TPR Linker.

PG16-TPR12-3BNC60, a C-to-C linked hetero-diFab containing 12 consensus tetratricopeptide-repeat (TPR) domains (Kajander et al., 2007, supra) as a protein linker (Klein et al., 2014, supra), was prepared from modified PG16 and 3BNC60 Fabs using a combination of sortase-catalyzed peptide ligation and click chemistry (Witte et al., 2013). The C-terminus of the PG16 Fab heavy chain was modified to include the amino acid sequence GGGGASLPETGGLNDIFEAQKIEWHEHHHHHH (SEQ ID NO: 42), comprising a flexible linker, the recognition sequence for S. aureus Sortase A (underlined), a BirA tag, and a 6×-His tag. The C-terminus of The 3BNC60 Fab heavy chain C-terminus was modified to include a (Gly₄Ser)₃ linker followed by 12 tandem TPR domains and the amino acid sequence ASGGGGSGGGGSGGGGSLPETGGHHHHHH (SEQ ID NO: 43), comprising a second (Gly₄Ser)₃ linker, the Sortase A recognition sequence (underlined), and a 6×-His tag. The Fabs were expressed in HEK-6E cells and purified with Ni-NTA and gel filtration chromatography as described in this disclosure. Peptides (GGGK with C-terminal azide and cyclooctyne click handles) were synthesized by GenScript, and sortase-catalyzed peptide ligation was used to attach the azide-containing peptide to PG16 Fab and the cyclooctyne-containing peptide to the 3BNC60-TPR12 fusion protein as described in Guimaraes et al., 2013, Nat. Protoc. 8:1787-1799, the entire contents of which are herein incorporated by reference. Approximate yields after each sortase reaction were approximately 30%. Peptide-ligated PG16 and 3BNC60 Fabs were passed over a Ni-NTA column to remove His-tagged enzyme and Fabs that did not lose their His tags during the reaction, mixed at equimolar ratios, and the click reaction was accomplished by incubating overnight at 25° C. The yield for the click reaction was approximately 65%. The resulting PG16-TPR12-3BNC60 hetero-diFab was purified by size exclusion chromatography to remove unreacted Fabs for an overall yield of approximately 22%.

Measurements of Intra-Spike Distances.

In order to derive predicted distances between two adjacent Fabs bound to HIV-1 Env, sFabs bound to their epitopes were superimposed on the structures of Env trimers in three different conformations: closed (a 4.7 Å crystal structure of a gp140 SOSIP trimer; PDB code 4NCO), open (a 9 Å EM structure of a SOSIP trimer-17b Fab complex; coordinates obtained from S. Subramaniam), partially-open (an ˜20 Å EM structure of a viral spike bound to b12 Fab; PDB code 3DNL). (Tran et al., 2012, PLoS pathog 8: e1002797, the entire contents of which are herein incorporated by reference.) The positions of the C_{H}1 and C_{L} domains in Fab structures used for docking were adjusted to create Fabs with the average elbow bend angle found in a survey of human Fab structures. The V_{H}-V_{L} domains of the adjusted Fabs were then superimposed on crystal structures of Fab-gp120 or Fab-gp140 complexes (PDB codes 3NGB, 2NY7 and 4CNO for complexes with VRC01, b12 and PGT122 Fabs, respectively) or a PG16-epitope scaffold complex (PDB code 4DQO). The position on Env trimer of 10-1074, a clonal variant of the PGT121-PGT123 family, was approximated using the 4CNO gp140-PGT122 structure. (Mouquet et al., 2012, Nature, 467:591-595, the entire contents of which are herein incorporated by reference.) In other cases, related antibodies, e.g., PG9/PG16 and VRC01/3BNC117/3BNC60, were also assumed to bind similarly. The complex structures were superimposed on the Env trimer structures by aligning the common portions. The distance between the Cys233_{heavy chain} carbon-α atoms of adjacent Fabs was then measured using PyMol to approximate the length of dsDNA bridges attached to Cys233_{heavy chain}. (Schrödinger, 2011, The PyMOL Molecular Graphics System (The PyMOL Molecular Graphics System, the entire contents of which are herein incorporated by reference.) Measurements derived using other EM structures for the closed and open trimers (PDB codes 3DNN, 3J5M and 3DNO) or using a recent 3.5 Å Env trimer crystal structure resulted in differences less than or equal to (≦) 10 Å for analogous distance measurements. (Pancera et al., 2014, Nature, 514:455-461, the entire contents of which are herein incorporated by reference.)

In Vitro Neutralization Assays.

Neutralization of pseudoviruses derived from primary HIV-1 isolates was monitored by the reduction of HIV-1 Tat-induced luciferase reporter gene expression in the presence of a single round of pseudovirus infection in TZM-bl cells as described in this disclosure and previously in Montefiori, 2005, supra).

Simulation of Fab and IgG Saturation of Surface-Bound Antigens.

Numerical analysis (using Mathematica, v. 10 was used to simulate saturation of surface-bound antigens by monovalent Fabs (Equation 1), bivalent IgGs to unpaired antigen (Ag) (Equation 2), and paired antigen (pAg) (Equations 3,4), where “paired antigen” was defined as antigens that are spaced such that an IgG can bind two epitopes simultaneously (e.g., intra-spike crosslinking of two epitopes on the same viral spike or inter-spike crosslinking between two viral spikes). In the bivalent model (Equations 3,4), the surface concentrations of antigen and IgG-antigen complexes were approximated by the inverse of the volume of a sphere (Vₛ) with radius equal to the hydrodynamic radius of the molecule multiplied by Avogadro's number (Nₐ) as described previously (Miller et al., 1998).

Fab binding to antigen:

$$Fab + Ag \begin{matrix}\to \\ ←\end{matrix} Fab - Ag$$

$$\frac{d [ Fab - Ag ]}{d t} = {k}_{a} [ Fab ] [ Ag ] - {k}_{d} [ Fab - Ag ] \qquad [ Equation 1 ]$$

IgG binding to unpaired antigen:

$$IgG + Ag \begin{matrix}\to \\ ←\end{matrix} IgG - Ag$$

$$\frac{d [ IgG - Ag ]}{d t} = 2 {k}_{a} [ IgG ] [ Ag ] - {k}_{d} [ IgG - Ag ] \qquad [ Equation 2 ]$$

IgG binding to paired antigen:

$$IgG + pAg \begin{matrix}\to \\ ←\end{matrix} IgG - pAg$$

$$IgG - pAg + pAg \begin{matrix}\to \\ ←\end{matrix} IgG - {pAg}_{2}$$

$$\begin{matrix}\frac{d [ IgG - pAg ]}{d t} = 2 {k}_{a} [ IgG ] [ pAg ] - {k}_{d} [ IgG - pAg ] \frac{1}{{V}_{s} {N}_{a}} - \frac{d [ IgG - {pAg}_{2} ]}{d t} & [ Equation 3 ] \\ \frac{d [ IgG - {pAg}_{2} ]}{d t} = {k}_{a} [ IgG - pAg ] \frac{1}{{V}_{T} {N}_{a}} [ pAg ] \frac{1}{{V}_{S} {N}_{a}} - 2 {k}_{d} [ IgG - {pAg}_{2} ] \frac{1}{{V}_{S} {N}_{a}} & [ Equation 4 ]\end{matrix}$$

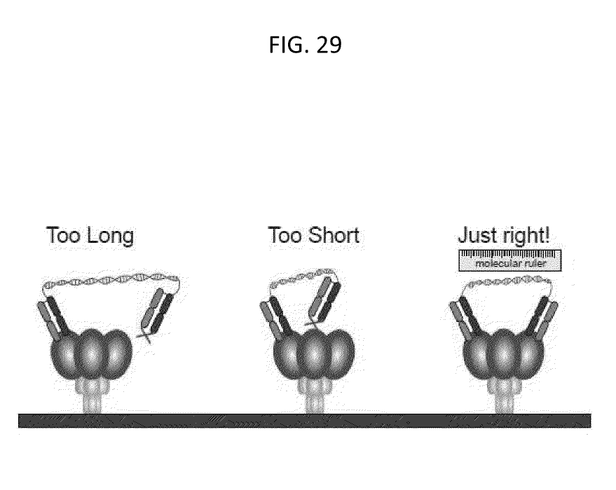

As disclosed throughout, for example in FIGS. 11A-11D and 15-21 anti-HIV-1 antibody Fabs are crosslinked to form homo-diFabs or hetero-diFabs having improved potency and neutralization. Analysis with varying lengths of dsDNA linkers demonstrated effective linker lengths for each of the anti-HIV-1 homo-diFabs and hetero-diFabs (FIG. 29). Using the dsDNA linker lengths, protein linker molecules of varying lengths are conjugated to the anti-HIV-1 antibody Fabs forming anti-HIV-1 compositions having improved viral potency.

While the present invention has been illustrated and described with reference to certain exemplary embodiments, those of ordinary skill in the art will understand that various modifications and changes may be made to the described embodiments without departing from the spirit and scope of the present invention, as defined in the following claims.

SEQUENCE LISTING

V1V2 PDB 4DQO for PG16
Heavy chain SEQ ID NO: 27:
PG16 HC: (4DQO)
PCA GLU GLN LEU VAL GLU SER GLY GLY GLY VAL VAL GLN
PRO GLY GLY SER LEU ARG LEU SER CYS LEU ALA SER GLY
PHE THR PHE HIS LYS TYR GLY MET HIS TRP VAL ARG GLN
ALA PRO GLY LYS GLY LEU GLU TRP VAL ALA LEU ILE SER
ASP ASP GLY MET ARG LYS TYR HIS SER ASP SER MET TRP
GLY ARG VAL THR ILE SER ARG ASP ASN SER LYS ASN THR
LEU TYR LEU GLN PHE SER SER LEU LYS VAL GLU ASP THR
ALA MET PHE PHE CYS ALA ARG GLU ALA GLY GLY PRO ILE
TRP HIS ASP ASP VAL LYS TYR TYS ASP PHE ASN ASP GLY
TYR TYR ASN TYR HIS TYR MET ASP VAL TRP GLY LYS GLY
THR THR VAL THR VAL SER SER ALA SER THR LYS GLY PRO
SER VAL PHE PRO LEU ALA PRO SER SER LYS SER THR SER
GLY GLY THR ALA ALA LEU GLY CYS LEU VAL LYS ASP TYR
PHE PRO GLU PRO VAL THR VAL SER TRP ASN SER GLY ALA
LEU THR SER GLY VAL HIS THR PHE PRO ALA VAL LEU GLN
SER SER GLY LEU TYR SER LEU SER SER VAL VAL THR VAL
PRO SER SER SER LEU GLY THR GLN THR TYR ILE CYS ASN
VAL ASN HIS LYS PRO SER ASN THR LYS VAL ASP LYS ARG
VAL GLU PRO LYS SER CYS GLY LEU GLU VAL LEU PHE
Light chain 4DQO: SEQ ID NO: 28:
PG16 LC:
GLN SER ALA LEU THR GLN PRO ALA SER VAL SER GLY SER
PRO GLY GLN THR ILE THR ILE SER CYS GLN GLY THR SER
SER ASP VAL GLY GLY PHE ASP SER VAL SER TRP TYR GLN
GLN SER PRO GLY LYS ALA PRO LYS VAL MET VAL PHE ASP
VAL SER HIS ARG PRO SER GLY ILE SER ASN ARG PHE SER
GLY SER LYS SER GLY ASN THR ALA SER LEU THR ILE SER
GLY LEU HIS ILE GLU ASP GLU GLY ASP TYR PHE CYS SER
SER LEU THR ASP ARG SER HIS ARG ILE PHE GLY GLY GLY
THR LYS VAL THR VAL LEU GLY GLN PRO LYS ALA ALA PRO
SER VAL THR LEU PHE PRO PRO SER SER GLU GLU LEU GLN
ALA ASN LYS ALA THR LEU VAL CYS LEU ILE SER ASP PHE
TYR PRO GLY ALA VAL THR VAL ALA TRP LYS ALA ASP SER
SER PRO VAL LYS ALA GLY VAL GLU THR THR THR PRO SER
LYS GLN SER ASN ASN LYS TYR ALA ALA SER SER TYR LEU
SER LEU THR PRO GLU GLN TRP LYS SER HIS LYS SER TYR
SER CYS GLN VAL THR HIS GLU GLY SER THR VAL GLU LYS
THR VAL ALA PRO THR GLU CYS SER
V1V2 Fab PDB 3U2S for PG9
heavy chain: SEQ ID NO: 29:
PG9 HC: (3U2S)
PCA ARG LEU VAL GLU SER GLY GLY GLY VAL VAL GLN PRO
GLY SER SER LEU ARG LEU SER CYS ALA ALA SER GLY PHE
ASP PHE SER ARG GLN GLY MET HIS TRP VAL ARG GLN ALA
PRO GLY GLN GLY LEU GLU TRP VAL ALA PHE ILE LYS TYR
ASP GLY SER GLU LYS TYR HIS ALA ASP SER VAL TRP GLY
ARG LEU SER ILE SER ARG ASP ASN SER LYS ASP THR LEU
TYR LEU GLN MET ASN SER LEU ARG VAL GLU ASP THR ALA
THR TYR PHE CYS VAL ARG GLU ALA GLY GLY PRO ASP TYR
ARG ASN GLY TYR ASN TYS TYS ASP PHE TYR ASP GLY TYR
TYR ASN TYR HIS TYR MET ASP VAL TRP GLY LYS GLY THR
THR VAL THR VAL SER SER ALA SER THR LYS GLY PRO SER
VAL PHE PRO LEU ALA PRO SER SER LYS SER THR SER GLY
GLY THR ALA ALA LEU GLY CYS LEU VAL LYS ASP TYR PHE
PRO GLU PRO VAL THR VAL SER TRP ASN SER GLY ALA LEU
THR SER GLY VAL HIS THR PHE PRO ALA VAL LEU GLN SER
SER GLY LEU TYR SER LEU SER SER VAL VAL THR VAL PRO
SER SER SER LEU GLY THR GLN THR TYR ILE CYS ASN VAL
ASN HIS LYS PRO SER ASN THR LYS VAL ASP LYS LYS VAL
GLU PRO LYS SER CYS ASP LYS GLY LEU GLU VAL LEU PHE
GLN
PG9 light chain: SEQ ID NO: 30
PG9 LC:
GLN SER ALA LEU THR GLN PRO ALA SER VAL SER GLY SER
PRO GLY GLN SER ILE THR ILE SER CYS GLN GLY THR SER
ASN ASP VAL GLY GLY TYR GLU SER VAL SER TRP TYR GLN
GLN HIS PRO GLY LYS ALA PRO LYS VAL VAL ILE TYR ASP
VAL SER LYS ARG PRO SER GLY VAL SER ASN ARG PHE SER
GLY SER LYS SER GLY ASN THR ALA SER LEU THR ILE SER
GLY LEU GLN ALA GLU ASP GLU GLY ASP TYR TYR CYS LYS
SER LEU THR SER THR ARG ARG ARG VAL PHE GLY THR GLY
THR LYS LEU THR VAL LEU GLY GLN PRO LYS ALA ALA PRO
SER VAL THR LEU PHE PRO PRO SER SER GLU GLU LEU GLN
ALA ASN LYS ALA THR LEU VAL CYS LEU ILE SER ASP PHE
TYR PRO GLY ALA VAL THR VAL ALA TRP LYS ALA ASP SER
SER PRO VAL LYS ALA GLY VAL GLU THR THR THR PRO SER
LYS GLN SER ASN ASN LYS TYR ALA ALA SER SER TYR LEU
SER LEU THR PRO GLU GLN TRP LYS SER HIS LYS SER TYR
SER CYS GLN VAL THR HIS GLU GLY SER THR VAL GLU LYS
THR VAL ALA PRO THR GLU CYS SER
CD4 PDB 3RPI for 3BNC60
heavy chain: SEQ ID NO: 31:
3BNC60 HC: (3RPI)
GLN VAL HIS LEU SER GLN SER GLY ALA ALA VAL THR LYS
PRO GLY ALA SER VAL ARG VAL SER CYS GLU ALA SER GLY
TYR LYS ILE SER ASP HIS PHE ILE HIS TRP TRP ARG GLN
ALA PRO GLY GLN GLY LEU GLN TRP VAL GLY TRP ILE ASN
PRO LYS THR GLY GLN PRO ASN ASN PRO ARG GLN PHE GLN
GLY ARG VAL SER LEU THR ARG GLN ALA SER TRP ASP PHE
ASP THR TYR SER PHE TYR MET ASP LEU LYS ALA VAL ARG
SER ASP ASP THR ALA ILE TYR PHE CYS ALA ARG GLN ARG
SER ASP PHE TRP ASP PHE ASP VAL TRP GLY SER GLY THR
GLN VAL THR VAL SER SER ALA SER THR LYS GLY PRO SER
VAL PHE PRO LEU ALA PRO SER SER LYS SER THR SER GLY
GLY THR ALA ALA LEU GLY CYS LEU VAL LYS ASP TYR PHE
PRO GLU PRO VAL THR VAL SER TRP ASN SER GLY ALA LEU
THR SER GLY VAL HIS THR PHE PRO ALA VAL LEU GLN SER
SER GLY LEU TYR SER LEU SER SER VAL VAL THR VAL PRO
SER SER SER LEU GLY THR GLN THR TYR ILE CYS ASN VAL
ASN HIS LYS PRO SER ASN THR LYS VAL ASP LYS ARG VAL
GLU PRO LYS SER CYS ASP LYS THR
CD4 light chain: SEQ ID NO: 32:
3BNC60 LC:
ASP ILE GLN MET THR GLN SER PRO SER SER LEU SER ALA
ARG VAL GLY ASP THR VAL THR ILE THR CYS GLN ALA ASN
GLY TYR LEU ASN TRP TYR GLN GLN ARG ARG GLY LYS ALA
PRO LYS LEU LEU ILE TYR ASP GLY SER LYS LEU GLU ARG
GLY VAL PRO ALA ARG PHE SER GLY ARG ARG TRP GLY GLN
GLU TYR ASN LEU THR ILE ASN ASN LEU GLN PRO GLU ASP
VAL ALA THR TYR PHE CYS GLN VAL TYR GLU PHE ILE VAL
PRO GLY THR ARG LEU ASP LEU LYS ARG THR VAL ALA ALA
PRO SER VAL PHE ILE PHE PRO PRO SER ASP GLU GLN LEU
LYS SER GLY THR ALA SER VAL VAL CYS LEU LEU ASN ASN
PHE TYR PRO ARG GLU ALA LYS VAL GLN TRP LYS VAL ASP
ASN ALA LEU GLN SER GLY ASN SER GLN GLU SER VAL THR
GLU GLN ASP SER LYS ASP SER THR TYR SER LEU SER SER
THR LEU THR LEU SER LYS ALA ASP TYR GLU LYS HIS LYS
VAL TYR ALA CYS GLU VAL THR HIS GLN GLY LEU SER SER
PRO VAL THR LYS SER PHE ASN ARG GLY GLU CYS
CD4 PDB 2NY7 for b12
heavy chain: SEQ ID NO: 33:
b12 HC: (2NY7)
GLN VAL GLN LEU VAL GLN SER GLY ALA GLU VAL LYS LYS
PRO GLY ALA SER VAL LYS VAL SER CYS GLN ALA SER GLY
TYR ARG PHE SER ASN PHE VAL ILE HIS TRP VAL ARG GLN
ALA PRO GLY GLN ARG PHE GLU TRP MET GLY TRP ILE ASN
PRO TYR ASN GLY ASN LYS GLU PHE SER ALA LYS PHE GLN
ASP ARG VAL THR PHE THR ALA ASP THR SER ALA ASN THR
ALA TYR MET GLU LEU ARG SER LEU ARG SER ALA ASP THR
ALA VAL TYR TYR CYS ALA ARG VAL GLY PRO TYR SER TRP
ASP ASP SER PRO GLN ASP ASN TYR TYR MET ASP VAL TRP
GLY LYS GLY THR THR VAL ILE VAL SER SER ALA SER THR
LYS GLY PRO SER VAL PHE PRO LEU ALA PRO SER SER LYS
SER THR SER GLY GLY THR ALA ALA LEU GLY CYS LEU VAL
LYS ASP TYR PHE PRO GLU PRO VAL THR VAL SER TRP ASN
SER GLY ALA LEU THR SER GLY VAL HIS THR PHE PRO ALA
VAL LEU GLN SER SER GLY LEU TYR SER LEU SER SER VAL
VAL THR VAL PRO SER SER SER LEU GLY THR GLN THR TYR
ILE CYS ASN VAL ASN HIS LYS PRO SER ASN THR LYS VAL
ASP LYS LYS ALA GLU PRO LYS SER CYS
CD4 light chain: SEQ ID NO: 34:
b12 LC:
GLU ILE VAL LEU THR GLN SER PRO GLY THR LEU SER LEU
SER PRO GLY GLU ARG ALA THR PHE SER CYS ARG SER SER
HIS SER ILE ARG SER ARG ARG VAL ALA TRP TYR GLN HIS
LYS PRO GLY GLN ALA PRO ARG LEU VAL ILE HIS GLY VAL
SER ASN ARG ALA SER GLY ILE SER ASP ARG PHE SER GLY
SER GLY SER GLY THR ASP PHE THR LEU THR ILE THR ARG
VAL GLU PRO GLU ASP PHE ALA LEU TYR TYR CYS GLN VAL
TYR GLY ALA SER SER TYR THR PHE GLY GLN GLY THR LYS
LEU GLU ARG LYS ARG THR VAL ALA ALA PRO SER VAL PHE
ILE PHE PRO PRO SER ASP GLU GLN LEU LYS SER GLY THR
ALA SER VAL VAL CYS LEU LEU ASN ASN PHE TYR PRO ARG
GLU ALA LYS VAL GLN TRP LYS VAL ASP ASN ALA LEU GLN
SER GLY ASN SER GLN GLU SER VAL THR GLU GLN ASP SER
LYS ASP SER THR TYR SER LEU SER SER THR LEU THR LEU
SER LYS ALA ASP TYR GLU LYS HIS LYS VAL TYR ALA CYS
GLU VAL THR HIS GLN GLY LEU ARG SER PRO VAL THR LYS
SER PHE ASN ARG GLY GLU CYS
V3 PDB 4FQ2 for 10-1074
heavy chain: SEQ ID NO: 35:
10-1074 HC:
GLN VAL GLN LEU GLN GLU SER GLY PRO GLY LEU VAL LYS
PRO SER GLU THR LEU SER VAL THR CYS SER VAL SER GLY
ASP SER MET ASN ASN TYR TYR TRP THR TRP ILE ARG GLN
SER PRO GLY LYS GLY LEU GLU TRP ILE GLY TYR ILE SER
ASP ARG GLU SER ALA THR TYR ASN PRO SER LEU ASN SER
ARG VAL VAL ILE SER ARG ASP THR SER LYS ASN GLN LEU
SER LEU LYS LEU ASN SER VAL THR PRO ALA ASP THR ALA
VAL TYR TYR CYS ALA THR ALA ARG ARG GLY GLN ARG ILE
TYR GLY VAL VAL SER PHE GLY GLU PHE PHE TYR TYR TYR
SER MET ASP VAL TRP GLY LYS GLY THR THR VAL THR VAL
SER SER ALA SER THR LYS GLY PRO SER VAL PHE PRO LEU
ALA PRO SER SER LYS SER THR SER GLY GLY THR ALA ALA
LEU GLY CYS LEU VAL LYS ASP TYR PHE PRO GLU PRO VAL
THR VAL SER TRP ASN SER GLY ALA LEU THR SER GLY VAL
HIS THR PHE PRO ALA VAL LEU GLN SER SER GLY LEU TYR
SER LEU SER SER VAL VAL THR VAL PRO SER SER SER LEU
GLY THR GLN THR TYR ILE CYS ASN VAL ASN HIS LYS PRO
SER ASN THR LYS VAL ASP LYS ARG VAL GLU PRO LYS SER
CYS ASP
light chain: SEQ ID NO: 36:
10-1074 LC:
SER TYR VAL ARG PRO LEU SER VAL ALA LEU GLY GLU THR
ALA ARG ILE SER CYS GLY ARG GLN ALA LEU GLY SER ARG
ALA VAL GLN TRP TYR GLN HIS ARG PRO GLY GLN ALA PRO
ILE LEU LEU ILE TYR ASN ASN GLN ASP ARG PRO SER GLY
ILE PRO GLU ARG PHE SER GLY THR PRO ASP ILE ASN PHE
GLY THR ARG ALA THR LEU THR ILE SER GLY VAL GLU ALA
GLY ASP GLU ALA ASP TYR TYR CYS HIS MET TRP ASP SER
ARG SER GLY PHE SER TRP SER PHE GLY GLY ALA THR ARG
LEU THR VAL LEU GLY GLN PRO LYS ALA ALA PRO SER VAL
THR LEU PHE PRO PRO SER SER GLU GLU LEU GLN ALA ASN
LYS ALA THR LEU VAL CYS LEU ILE SER ASP PHE TYR PRO
GLY ALA VAL THR VAL ALA TRP LYS ALA ASP SER SER PRO
VAL LYS ALA GLY VAL GLU THR THR THR PRO SER LYS GLN
SER ASN ASN LYS TYR ALA ALA SER SER TYR LEU SER LEU
THR PRO GLU GLN TRP LYS SER HIS ARG SER TYR SER CYS
GLN VAL THR HIS GLU GLY SER THR VAL GLU LYS THR VAL
ALA PRO THR GLU CYS SER
V3 PDB 4FQ1 for PGT121
heavy chain: SEQ ID NO: 37:
PGT121 HC:
GLN MET GLN LEU GLN GLU SER GLY PRO GLY LEU VAL LYS
PRO SER GLU THR LEU SER LEU THR CYS SER VAL SER GLY
ALA SER ILE SER ASP SER TYR TRP SER TRP ILE ARG ARG
SER PRO GLY LYS GLY LEU GLU TRP ILE GLY TYR VAL HIS
LYS SER GLY ASP THR ASN TYR SER PRO SER LEU LYS SER
ARG VAL ASN LEU SER LEU ASP THR SER LYS ASN GLN VAL
SER LEU SER LEU VAL ALA ALA THR ALA ALA ASP SER GLY
LYS TYR TYR CYS ALA ARG THR LEU HIS GLY ARG ARG ILE
TYR GLY ILE VAL ALA PHE ASN GLU TRP PHE THR TYR PHE
TYR MET ASP VAL TRP GLY ASN GLY THR GLN VAL THR VAL
SER SER ALA SER THR LYS GLY PRO SER VAL PHE PRO LEU
ALA PRO SER SER LYS SER THR SER GLY GLY THR ALA ALA
LEU GLY CYS LEU VAL LYS ASP TYR PHE PRO GLU PRO VAL
THR VAL SER TRP ASN SER GLY ALA LEU THR SER GLY VAL
HIS THR PHE PRO ALA VAL LEU GLN SER SER GLY LEU TYR
SER LEU SER SER VAL VAL THR VAL PRO SER SER SER LEU
GLY THR GLN THR TYR ILE CYS ASN VAL ASN HIS LYS PRO
SER ASN THR LYS VAL ASP LYS ARG VAL GLU PRO LYS SER
CYS ASP
V3 light chain: SEQ ID NO: 38
PGT121 LC:
SER ASP ILE SER VAL ALA PRO GLY GLU THR ALA ARG ILE
SER CYS GLY GLU LYS SER LEU GLY SER ARG ALA VAL GLN
TRP TYR GLN HIS ARG ALA GLY GLN ALA PRO SER LEU ILE
ILE TYR ASN ASN GLN ASP ARG PRO SER GLY ILE PRO GLU
ARG PHE SER GLY SER PRO ASP SER PRO PHE GLY THR THR
ALA THR LEU THR ILE THR SER VAL GLU ALA GLY ASP GLU
ALA ASP TYR TYR CYS HIS ILE TRP ASP SER ARG VAL PRO
THR LYS TRP VAL PHE GLY GLY GLY THR THR LEU THR VAL
LEU GLY GLN PRO LYS ALA ALA PRO SER VAL THR LEU PHE
PRO PRO SER SER GLU GLU LEU GLN ALA ASN LYS ALA THR
LEU VAL CYS LEU ILE SER ASP PHE TYR PRO GLY ALA VAL
THR VAL ALA TRP LYS ALA ASP SER SER PRO VAL LYS ALA
GLY VAL GLU THR THR THR PRO SER LYS GLN SER ASN ASN
LYS TYR ALA ALA SER SER TYR LEU SER LEU THR PRO GLU
GLN TRP LYS SER HIS ARG SER TYR SER CYS GLN VAL THR
HIS GLU GLY SER THR VAL GLU LYS THR VAL ALA PRO THR
GLU CYS SER
gp41 4G6F for 10E8
heavy chain: SEQ ID NO: 39:
10E8 HC: (4G6F)
GLU VAL GLN LEU VAL GLU SER GLY GLY GLY LEU VAL LYS
PRO GLY GLY SER LEU ARG LEU SER CYS SER ALA SER GLY
PHE ASP PHE ASP ASN ALA TRP MET THR TRP VAL ARG GLN
PRO PRO GLY LYS GLY LEU GLU TRP VAL GLY ARG ILE THR
GLY PRO GLY GLU GLY TRP SER VAL ASP TYR ALA ALA PRO
VAL GLU GLY ARG PHE THR ILE SER ARG LEU ASN SER ILE
ASN PHE LEU TYR LEU GLU MET ASN ASN LEU ARG MET GLU
ASP SER GLY LEU TYR PHE CYS ALA ARG THR GLY LYS TYR
TYR ASP PHE TRP SER GLY TYR PRO PRO GLY GLU GLU TYR
PHE GLN ASP TRP GLY ARG GLY THR LEU VAL THR VAL SER
SER ALA SER THR LYS GLY PRO SER VAL PHE PRO LEU ALA
PRO SER SER LYS SER THR SER GLY GLY THR ALA ALA LEU
GLY CYS LEU VAL LYS ASP TYR PHE PRO GLU PRO VAL THR
VAL SER TRP ASN SER GLY ALA LEU THR SER GLY VAL HIS
THR PHE PRO ALA VAL LEU GLN SER SER GLY LEU TYR SER
LEU SER SER VAL VAL THR VAL PRO SER SER SER LEU GLY
THR GLN THR TYR ILE CYS ASN VAL ASN HIS LYS PRO SER
ASN THR LYS VAL ASP LYS ARG VAL GLU PRO LYS SER CYS
ASP LYS
gp41 light chain: SEQ ID NO: 40:
10E8 LC:
SER TYR GLU LEU THR GLN GLU THR GLY VAL SER VAL ALA
LEU GLY ARG THR VAL THR ILE THR CYS ARG GLY ASP SER
LEU ARG SER HIS TYR ALA SER TRP TYR GLN LYS LYS PRO
GLY GLN ALA PRO ILE LEU LEU PHE TYR GLY LYS ASN ASN
ARG PRO SER GLY VAL PRO ASP ARG PHE SER GLY SER ALA
SER GLY ASN ARG ALA SER LEU THR ILE SER GLY ALA GLN
ALA GLU ASP ASP ALA GLU TYR TYR CYS SER SER ARG ASP
LYS SER GLY SER ARG LEU SER VAL PHE GLY GLY GLY THR
LYS LEU THR VAL LEU SER GLN PRO LYS ALA ALA PRO SER
VAL THR LEU PHE PRO PRO SER SER GLU GLU LEU GLN ALA
ASN LYS ALA THR LEU VAL CYS LEU ILE SER ASP PHE TYR
PRO GLY ALA VAL THR VAL ALA TRP LYS ALA ASP SER SER
PRO VAL LYS ALA GLY VAL GLU THR THR THR PRO SER LYS
GLN SER ASN ASN LYS TYR ALA ALA SER SER TYR LEU SER
LEU THR PRO GLU GLN TRP LYS SER HIS ARG SER TYR SER
CYS GLN VAL THR HIS GLU GLY SER THR VAL GLU LYS THR
VAL ALA PRO THR GLU CYS SER

What is claimed is:

1. A composition comprising:
a first anti-HIV-1 antibody Fab;
a second anti-HIV-1 antibody Fab; and
a linker molecule conjugated between the C-terminus of the first anti-HIV-1 antibody Fab and the C-terminus of the second anti-HIV-1 antibody Fab.

2. The composition of claim 1, wherein the linker molecule is selected from the group consisting of single stranded nucleic acids, double stranded nucleic acids, amino acids, and combinations thereof.

3. The composition of claim 1, wherein the first anti-HIV-1 antibody Fab and the second anti-HIV-1 antibody Fab are each independently selected from the group consisting of anti-gp120 V1V2 Fabs, anti-gp120 V3 Fabs, anti-gp120 CD4 Fabs, and anti-gp41 Fabs.

4. The composition of claim 3, wherein the anti-gp120 V1V2 Fab comprises:
a heavy chain comprising anti-gp120 V1V2 binding residues corresponding to 57-59, 61, 64, 100, 100B, 100D, 100E, 100F, 100G, 100H, 100I, 100J, 100K, 100L, 100O, 100P, 100Q, 100R according to PDB 4DQO; and
a light chain comprising gp120 V1V2 binding residues corresponding to 31, 32, 50, 91, 94, 95A according to PDB 4DQO.

5. The composition of claim 3, wherein the anti-gp120 V1V2 Fab comprises:
a heavy chain comprising anti-gp120 V1V2 binding residues corresponding to 31, 53, 55, 100, 100B, 100E, 100F, 100G, 100H, 100I, 100J, 100K, 100L, 100O, 100P, 100Q, 100R according to PDB 3U2S; and
a light chain comprising anti-gp120 V1V2 binding residues corresponding to 31, 32, 50, 91, 94, and 95A according to PDB 3U2S.

6. The composition of claim 3, wherein the anti-gp120 CD4 Fab comprises:
a heavy chain comprising anti-gp120 CD4 binding residues corresponding to 30, 47, 50, 53-58, 60, 61, 64, 71, 71D, 72, 98, and 100 according to PDB 4JPV; and
a light chain comprising anti-gp120 CD4 binding residues corresponding to 27, 32, 91, 96, and 97, according to PDB 4JPV.

7. The composition of claim 3, wherein the anti-gp120 CD4 Fab comprises:
a heavy chain comprising anti-gp120 CD4 binding residues corresponding to 28, 30-33, 52-54, 56, 96-100, 100G, and 100H according to PDB 2NY7.

8. The composition of claim 3, wherein the anti-gp41 Fab comprises:
a heavy chain comprising anti-gp41 CD4 binding residues corresponding to 28, 31, 33, 52, 52B, 52C, 53, 56, 97-99, 100A, 100B, 100D, 100E, 100F, and 100G according to PDB 4G6F; and
a light chain comprising anti-gp41 binding residue corresponding to 95B.

9. The composition of claim 1, wherein the first anti-HIV antibody Fab and the second anti-HIV antibody Fab are each modified at the C-terminus for conjugation to the linker molecule.

10. The composition of claim 1, wherein the linker molecule comprises:
a first nucleic acid comprising a first segment conjugated at its 5′ end to the first anti-HIV antibody Fab and conjugated at its 3′ end to a sense strand of DNA; and
a second nucleic acid comprising a second segment conjugated at its 5′ end to the second anti-HIV antibody Fab and conjugated at its 3′ end to an anti-sense strand of DNA complementary to the sense strand of DNA of the first nucleic acid.

11. The composition of claim 10, wherein the first nucleic acid further comprises a second segment conjugated to the 3′ end of the sense strand of DNA, and the second nucleic acid further comprises a second segment conjugated to the 3′ end of the anti-sense strand of DNA.

12. The composition of claim 10, wherein the sense strand of DNA and the anti-sense strand of DNA each have a length selected from the group consisting of 20 to 100 base pairs, 25 to 80 base pairs, 30 to 70 base pairs, and 40 to 60 base pairs.

13. The composition of claim 1, wherein the linker molecule comprises a pair of nucleic acids having a pair of sequences selected from the group consisting of SEQ ID Nos: 3 and 4; SEQ ID Nos: 5 and 6; SEQ ID Nos: 7 and 8; SEQ ID Nos: 9 and 10; SEQ ID Nos: 11 and 12; SEQ ID Nos: 13 and 14; SEQ ID Nos: 15 and 16; SEQ ID Nos: 17 and 18; and SEQ ID Nos: 19 and 20; SEQ ID Nos: 21 and 22; SEQ ID Nos: 23 and 24; and SEQ ID Nos: 25 and 26.

14. The composition of claim 1, wherein the linker molecule comprises from 3 tetratricopeptide repeat (TPR)(SEQ ID NO: 41) domains up to 30 TPR domains.

15. The composition of claim 1, wherein when the first anti-HIV-1 antibody Fab is anti-gp120 CD4 and the second anti-HIV-1 antibody Fab is anti-gp120 CD4, the linker molecule comprises from 12 TPR (SEQ ID NO: 41) domains to 20 TPR domains.

16. The composition of claim 1, wherein when the first anti-HIV-1 antibody Fab is anti-gp120 V1V2 and the second anti-HIV-1 antibody Fab is anti-gp120 V1V2, the linker molecule comprises from 18 TPR (SEQ ID NO: 41) domains to 30 TPR domains.

17. The composition of claim 1, wherein when the first anti-HIV-1 antibody Fab is anti-gp120 V3 and the second anti-HIV-1 antibody Fab is anti-gp120 V3, the linker molecule comprises from 6 TPR (SEQ ID NO: 41) domains to 12 TPR domains.

18. The composition of claim 1, wherein when the first anti-HIV-1 antibody Fab is anti-gp120 V1V2 and the second anti-HIV-1 antibody Fab is anti-gp120 CD4, the linker molecule comprises from 6 TPR (SEQ ID NO: 41) domains to 15 TPR domains.

19. The composition of claim 1, wherein when the first anti-HIV-1 antibody Fab is anti-gp120 V3 and the second anti-HIV-1 antibody Fab is anti-gp120 CD4, the linker molecule comprises from 6 TPR (SEQ ID NO: 41) domains to 18 TPR domains.

20. The composition of claim 1, wherein when the first anti-HIV-1 antibody Fab is anti-gp41 and the second anti-HIV-1 antibody Fab is anti-gp120 CD4, the linker molecule comprises from 6 TPR (SEQ ID NO: 41) domains to 21 TPR domains.
